(19) United States
(12) Patent Application Publication
(10) Pub. No.: US 2024/0325657 A1
(43) Pub. Date: Oct. 3, 2024

(54) Needle Guard Assembly for Fluid Removal From a Glass Ampoule

(71) Applicant: Becton, Dickinson and Company, Franklin Lakes, NJ (US)

(72) Inventors: Rahul Malviya, Salmon Arm (CA); Amarsinh Deeliprao Jadhav, Hyderabad (IN); Aman Desai, Bengaluru (IN); Kevin M. Ryan, Whitehouse Station, NJ (US); Pushkal Singh, Indore (IN); Shishir Prasad, Ramsey, NJ (US)

(21) Appl. No.: 18/613,659

(22) Filed: Mar. 22, 2024

Related U.S. Application Data
(60) Provisional application No. 63/456,082, filed on Mar. 31, 2023.

(51) Int. Cl. A61M 5/32

(52) U.S. Cl. CPC A61M 5/322; A61M 5/3204

(57) ABSTRACT

A needle guard assembly includes a needle cannula defining a lumen having a proximal end, a distal end with a sharpened tip, and a tubular body extending between the proximal end and the distal end. The needle guard assembly also includes a needle hub connected to the proximal end of the needle cannula configured to be mounted to a fluid delivery device to provide fluid communication between the fluid delivery device and the lumen of the needle cannula and a liquid-permeable cover member extending over at least the distal end of the needle cannula having a proximal end engaged to the needle hub and a closed distal end enclosing the distal tip of the needle cannula.

### CROSS-REFERENCE TO RELATED APPLICATION

The present application claims priority to U.S. Provisional Application No. 63/456,082 entitled “Needle Guard Assembly for Fluid Removal from a Glass Ampoule” filed Mar. 31, 2023, the entire disclosure of which is hereby incorporated by reference.

### BACKGROUND OF THE INVENTION

### Field of the Invention

The present disclosure relates generally to needle guard assemblies for syringes and other vascular access devices and, in particular, to a needle guard assembly comprising a liquid-permeable cover member for separating solid particles from a medical solution.

### Description of Related Art

Medical liquids and solutions for injection can be packaged in a variety of containers, enclosures, ampoules, receptacles, and vials made from different types of rigid materials. During a fluid injection procedure, the medical solution is drawn from the container into a syringe barrel and then delivered to the patient by injection through an injection needle (e.g., a hypodermic needle). Conventional syringes used for such injection procedures can comprise a barrel having an open proximal end and an opposed distal end. A distal tip, sometimes referred to as a luer or luer tip, projects from the distal end of the syringe barrel and includes a narrow passage or channel in fluid communication with an interior of the syringe barrel. The injection needle can be connected to the syringe through a luer connection between the distal tip and a needle hub of the injection needle.

In many cases, medical solutions are packaged in glass ampoules. To administer these medical solutions, a practitioner snaps a neck of the ampoule to access the medical solution contained therein. However, the process of snapping the glass neck can introduce glass shards into the medical solution. In order to prevent the glass shards from being drawn into the syringe barrel during a fluid injection procedure, a filter needle (also referred to as a blunt needle) can be used to extract the glass shards from the medical solution prior to preparing a fluid delivery device for the injection.

More specifically, in accordance with current methods, the practitioner inserts the filter needle into the ampoule through the open neck of the ampoule and draws the medical solution through the filter needle into a syringe barrel. The glass shards either adhere to the outer surface of the filter needle or remain in the ampoule. Once the medical solution is in the syringe barrel, the practitioner then removes the filter needle from the syringe and attaches an injection needle (e.g., a hypodermic needle) to a syringe, which is used for injecting the medical solution from the syringe barrel to the patient. However, this multi-needle process can be cumbersome and time consuming for practitioners. Also, there are multiple risks involved during this two-needle process including, for example, additional opportunities to introduce contamination to the medical solution or medical devices, as well as an increased possibility of needle sticks and treatment delays as the two needles are obtained and prepared for use.

### SUMMARY OF THE INVENTION

According to an aspect of the present disclosure, a needle guard assembly includes a needle cannula defining a lumen having a proximal end, a distal end with a sharpened tip, and a tubular body extending between the proximal end and the distal end. The needle guard assembly also includes a needle hub connected to the proximal end of the needle cannula configured to be mounted to a fluid delivery device to provide fluid communication between the fluid delivery device and the lumen of the needle cannula and a liquid-permeable cover member extending over at least the distal end of the needle cannula having a proximal end engaged to the needle hub and a closed distal end enclosing the distal tip of the needle cannula.

According to another aspect of the present disclosure, a fluid delivery device includes a barrel having an open proximal, a closed distal end with a distal tip, and a sidewall extending between the proximal end and the distal end. The fluid delivery device also includes the previously described needle guard assembly, which is mounted to the distal tip of the barrel such that the lumen of the needle cannula is in fluid communication with an interior of the barrel through the distal tip of the barrel. The fluid delivery device also includes a stopper in the barrel for expelling a medical solution from the barrel through the distal tip and to the lumen of the needle cannula.

According to another aspect of the present disclosure, a method of use for a fluid delivery device that includes the needle guard assembly, as previously described, includes a step of inserting the needle guard assembly with the cover member and the needle cannula into a medical fluid container. The method also includes steps of: drawing medical solution in the container through the cover member and into the lumen of the needle cannula; drawing the medical solution from the lumen of the needle cannula into a reservoir of the fluid delivery device; and removing the needle guard assembly from the container after a sufficient amount of the medical solution is collected in the reservoir.

Non-limiting illustrative examples of embodiments of the present disclosure will now be described in the following numbered clauses.

Clause 1: A needle guard assembly, comprising: a needle cannula defining a lumen comprising a proximal end, a distal end comprising a sharpened tip, and a tubular body extending between the proximal end and the distal end; a needle hub connected to the proximal end of the needle cannula configured to be mounted to a fluid delivery device to provide fluid communication between the fluid delivery device and the lumen of the needle cannula; and a liquid-permeable cover member extending over at least the distal end of the needle cannula comprising a proximal end engaged to the needle hub and a closed distal end enclosing the distal tip of the needle cannula.

Clause 2: The needle guard assembly of clause 1, wherein the liquid-permeable cover member is configured to filter solid particles from a medical solution before the medical solution is drawn into the lumen of the needle cannula.

Clause 3: The needle guard assembly of clause 2, wherein the solid particles comprise shards of glass.

Clause 4: The needle guard assembly of any of clauses 1-3, wherein the needle cannula comprises an injection needle for subcutaneous, intramuscular, and/or intravenous injections.

Clause 5: The needle guard assembly of any of clauses 1-4, wherein the cover member is substantially rigid defining a cylindrical cavity enclosing the sharpened tip of the needle cannula without contacting the needle cannula.

Clause 6: The needle guard assembly of any of clauses 1-5, wherein the cover member comprises a rigid thermoplastic, such as at least one of polycarbonate, high density polyethylene, polypropylene, polyether ketone (PEEK), polytetrafluoroethylene (PTFE), acrylonitrile butadiene styrene (ABS), biaxially-oriented polyethylene terephthalate (BOPET), and/or polyimide.

Clause 7: The needle guard assembly of clause 1, wherein the needle hub comprises a rigid thermoplastic, such as at least one of polycarbonate, high density polyethylene, polypropylene, polyether ketone (PEEK), polytetrafluoroethylene (PTFE), acrylonitrile butadiene styrene (ABS), biaxially-oriented polyethylene terephthalate (BOPET), and/or polyimide.

Clause 8: The needle guard assembly of any of clauses 1-7, wherein the needle comprises stainless steel, a rigid thermoplastic polymer, or a bioabsorbable rigid polymer.

Clause 9: The needle guard assembly of any of clauses 1-8, wherein the cover member comprises a plurality of perforations for permitting solutions to pass through the cover member and into the lumen of the needle cannula.

Clause 10: The needle guard assembly of clause 9, wherein the perforations comprise holes that are less than or equal to 5 microns in diameter, or preferably from about 1 micron to about 5 microns in diameter.

Clause 11: The needle guard assembly of clause 9 or clause 10, wherein the perforations comprise holes made by laser cutting or heat staking.

Clause 12: The needle guard assembly of any of clauses 9-11, wherein the perforations comprise a first plurality of holes extending through a sidewall of the cover member and a second plurality of holes extending through the closed distal end of the cover member.

Clause 13: The needle guard assembly of clause 12, wherein the first plurality of holes are arranged in a plurality of axially aligned groups extending along the sidewall of the cover member, and wherein the second plurality of holes are arranged as a plurality of radially extending groups extending from a common point.

Clause 14: The needle guard assembly of any of clauses 1-13, wherein the cover member comprises ridges extending radially outward from an outer surface of the cover member forming a gripping surface of the cover member.

Clause 15: The needle guard assembly of any of clauses 1-14, wherein the proximal end of the cover member slides over and is frictionally engaged to an outer surface of the needle hub.

Clause 16: The needle guard assembly of clause 15, wherein the needle hub comprises at least one raised protrusion on a portion of the outer surface of the hub engaged by the cover member to enhance the connection between the needle hub and the cover member.

Clause 17: The needle guard assembly of any of clauses 1-16, further comprising an outer shield configured to be positioned over the cover member for protecting the cover member during transportation and before the use.

Clause 18: The needle guard assembly of clause 17, wherein the outer shield comprises a proximal portion configured to engage a portion of the outer surface of the needle hub for securing the outer shield to the needle hub.

Clause 19: The needle guard assembly of clause 18, wherein the portion of the outer surface of the needle hub contacted by the outer shield is proximal to a portion of the outer surface of the needle hub contacted by the cover member.

Clause 20: The needle guard assembly of any of clauses 17-19, wherein the cover member and the outer shield comprise a rigid thermoplastic comprising at least one of polycarbonate, high density polyethylene, polypropylene, polyether ketone (PEEK), polytetrafluoroethylene (PTFE), acrylonitrile butadiene styrene (ABS), biaxially-oriented polyethylene terephthalate (BOPET), and/or polyimide.

Clause 21: The needle guard assembly of any of clauses 1-21, wherein the cover member is tiered comprising at least a narrow distal tier and a wide proximal tier, and wherein perforations are on the distal tier, with the proximal tier being free from perforations.

Clause 22: The needle guard assembly of clause 21, wherein an inner surface of the proximal tier engages the needle hub by a friction engagement.

Clause 23: The needle guard assembly of clause 21 or clause 22, further comprising a seal positioned in the cover member comprising a central opening sized to receive the sidewall of the needle cannula.

Clause 24: The needle guard assembly of clause 23, wherein the seal is seated at an intersection between the proximal tier and the distal tier, and wherein the seal allows for creation of suction in a space defined by the seal and the cover member during aspiration of medical solution into the lumen of the needle cannula.

Clause 25: The needle guard assembly of any of clauses 20-24, further comprising a removable cap positioned over perforations of the cover member.

Clause 26: The needle guard assembly of clause 25, wherein the cap comprises a closed distal end sized to receive the closed distal end of the cover member, an outer proximal end, and a sidewall extending between the distal end and the proximal end, and wherein the sidewall of the cap contacts an outer surface of the cover member forming a friction engagement with the cover member for retaining the cap on the cover member.

Clause 27: The needle guard assembly of any of clauses 1-26, wherein the cover member comprises a cap comprising an open proximal end, a closed distal end, and a cylindrical sidewall extending therebetween, and at least one arm mounted between the needle hub and the cap for moving the cap between an initial position, where the cap covers the distal end of the needle cannula, and a retracted position where the cap is positioned on the sidewall of needle cannula proximal to the distal end of the needle cannula, thereby exposing the sharpened tip of the needle cannula.

Clause 28: The needle guard assembly of any of clauses 1-27, wherein the cover member comprises a cap comprising an open proximal end, a closed distal end, and a cylindrical sidewall extending therebetween, and a first arm and a second arm mounted between the needle hub and the cap for moving the cap along the needle cannula between an initial position, where the cap covers the distal end of the needle cannula, and a retracted position where the cap is positioned on the sidewall of needle cannula proximal to the distal end of the needle cannula, thereby exposing the sharpened tip of the needle cannula.

Clause 29: The needle guard assembly of clause 28, wherein the cap slides along the needle cannula between the initial position and the retracted position.

Clause 30: The needle guard assembly of clause 28 or clause 29, wherein a first arm and a second arm each comprise a proximal segment hingedly joined to a distal segment, and wherein the distal segments bend radially inwardly relative to the proximal segments as the first arm and the second arm move from the initial position to the retracted position.

Clause 31: The needle guard assembly of clause 30, wherein the first arm and the second arm are biased to the retracted position, and wherein, in the retracted position, the distal segment of the first arm and/or the second arm is substantially perpendicular to a longitudinal axis of the needle guard.

Clause 32: The needle guard assembly of any of clauses 28-31, further comprising a retention ring positioned about the hub configured to engage a portion of the first arm or the second arm for retaining the cap over the distal end of the needle cannula.

Clause 33: The needle guard assembly of clause 32, wherein the retaining ring comprises hooks configured to engage the first arm and/or the second arm for maintaining the arms in the initial positon.

Clause 34: The needle guard assembly of clause 32 or clause 33, wherein, once disengaged from the retaining ring, the first arm and the second arm are biased to move the cap to the retracted position.

Clause 35: The needle guard assembly of any of clauses 28-34, wherein the cap comprises a pierceable distal end configured to be pierced by the sharpened tip of the needle cannula as the cap moves from the initial position to the retracted position.

Clause 36: The needle guard assembly of any of clauses 28-35, further comprising a receptacle comprising a plurality of elongated tines sized to receive the cap, wherein the receptacle is connected to the first arm and to the second arm to support the cap as the cap moves between the initial position and the retracted position.

Clause 37: The needle guard assembly of any of clauses 28-36, wherein the cap comprises a filter configured to retain solid particles from a medical solution passing through the cap to the lumen of the needle cannula.

Clause 38: The needle guard assembly of any of clauses 1-37, further comprising a cylindrical seal positioned at the distal end of the cover member, the cylindrical seal comprising a central channel comprising a proximal portion sized to receive the distal end of the needle cannula and a narrowed distal portion extending to an opening in the closed distal end of the cover member.

Clause 39: The needle guard assembly of clause 38, further comprising a filter positioned between the cylindrical seal and the closed distal end of the cover member, positioned so that medical solution pirated through the cover member passes through the filter and then into the lumen of the needle cannula.

Clause 40: The needle guard assembly of clause 39, wherein the filter comprises a flat sheet of filter paper having an outer diameter matching an inner diameter of the distal end of the cover member.

Clause 41: The needle guard assembly of clause 39 or clause 40, wherein the filter comprises a woven acrylic membrane disc.

Clause 42: The needle assembly of any of clauses 39-41, wherein the filter is configured to filter particles larger than about 5 microns.

Clause 43: The needle guard assembly of any of clauses 1-42, further comprising an elastomeric ring positioned about the outer surface of the tubular body of the needle cannula.

Clause 44: The needle guard assembly of clause 43, wherein the cover member comprises at least one slot configured to receive the elastomeric ring for retaining the ring within the cover member as the cover member is removed from the needle cannula, thereby removing the ring from the needle cannula.

Clause 45: The needle guard assembly of clause 44, wherein the cover member is configured such that the ring is received within the slot as the cover member is removed from the needle cannula, and the ring slides past the at least one slot without being received in the slot as the needle cannula is inserted into the cover member.

Clause 46: The needle guard assembly of clause 44 or clause 45, wherein the cover member further comprises at least one tab proximal to the slot for retaining the ring within the slot as the cover member is removed from the needle cannula.

Clause 47: The needle guard assembly of clause 46, wherein the tabs deflect radially outward as the needle cannula and ring attached thereto are inserted into the cover member.

Clause 48: A fluid delivery device comprising: a barrel comprising an open proximal, a closed distal end comprising a distal tip, and a sidewall extending between the proximal end and the distal end; the needle guard assembly of any of clauses 1-47, mounted to the distal tip of the barrel such that the lumen of the needle cannula is in fluid communication with an interior of the barrel through the distal tip of the barrel; and a stopper in the barrel for expelling a medical solution from the barrel through the distal tip and to the lumen of the needle cannula.

Clause 49: A method of use for a fluid delivery device comprising the needle guard assembly of any of clauses 1-47, the method comprising: inserting the needle guard assembly comprising the cover member and the needle cannula into a medical fluid container; drawing medical solution in the container through the cover member and into the lumen of the needle cannula; drawing the medical solution from the lumen of the needle cannula into a reservoir of the fluid delivery device; and removing the needle guard assembly from the container after a sufficient amount of the medical solution is collected in the reservoir.

Clause 50: The method of clause 49, wherein the medical fluid container comprises a glass ampoule containing the medical solution.

Clause 51: The method of clause 49 or clause 50, wherein the fluid delivery device comprises a syringe and the reservoir comprises a barrel of the syringe.

Clause 52: The method of clause 51, further comprising attaching the needle guard assembly to a distal tip of the syringe prior to inserting the needle guard assembly into the medical fluid container.

Clause 53: The method of any of clauses 49-52, further comprising removing an outer shield from the needle guard assembly prior to inserting the needle guard assembly into the medical fluid container.

Clause 54: The method of clauses 49-53, further comprising removing the cover member from the needle hub and needle cannula after removing the needle guard assembly from the medical fluid container, thereby exposing the sharpened tip of the needle cannula.

Clause 55: The method of clause 54, further comprising inserting the exposed sharpened tip into skin, muscle tissue, and/or vasculature of a patient.

Clause 56: The method of clauses 49-55, wherein the medical fluid container comprises a glass ampoule, the method further comprising breaking a narrow neck of the glass ampoule prior to inserting the needle guard assembly into the glass ampoule.

### BRIEF DESCRIPTION OF THE DRAWINGS

FIG. 1A is a perspective view of a needle guard assembly, according to an aspect of the present disclosure.

FIG. 1B is an exploded perspective view of the needle guard assembly of FIG. 1A.

FIG. 1C is a cross-sectional view of the needle guard assembly of FIG. 1A.

FIG. 1D is an enlarged cross-sectional view of a portion of the needle guard assembly of FIG. 1C.

FIG. 2A is a perspective view of the cover member of the needle guard assembly of FIG. 1A, according to an aspect of the present disclosure.

FIG. 2B is an enlarged perspective view of a portion of the cover member of FIG. 2A.

FIG. 3A is a cross-sectional view of another example of a needle guard assembly, according to an aspect of the present disclosure.

FIG. 3B is an enlarged cross-sectional view of a portion of the needle guard assembly of FIG. 3A.

FIG. 4A is a front view of the needle guard assembly of FIG. 1A attached to a fluid delivery device in a pre-use position, according to an aspect of the present disclosure.

FIG. 4B is a front view of the needle guard assembly and fluid delivery device of FIG. 4A with the needle guard assembly inserted into an ampoule.

FIG. 4C is a front view of the needle guard assembly and fluid delivery device of FIG. 4A with the needle guard removed after medical solution has been injected to a patient through the injection needle, according to an aspect of the present disclosure.

FIG. 5A is a perspective view of another example of a needle guard assembly, according to an aspect of the present disclosure.

FIG. 5B is a cross-sectional view of the needle guard assembly of FIG. 5A.

FIG. 5C is an exploded perspective view of the needle guard assembly of FIG. 5A.

FIG. 6A is a front view of the needle guard assembly of FIG. 5A attached to a fluid delivery device in a pre-use position, according to an aspect of the present disclosure.

FIG. 6B is a front view of the needle guard assembly and fluid delivery device of FIG. 6A with the needle guard assembly inserted into an ampoule, according to an aspect of the present disclosure.

FIG. 6C is a front view of the needle guard assembly and fluid delivery device of FIG. 6A with the needle guard removed after medical solution has been injected to a patient through the injection needle, according to an aspect of the present disclosure.

FIG. 7A is a cross-sectional view of another example of a needle guard assembly, according to an aspect of the present disclosure.

FIG. 7B is an exploded perspective view of the needle guard assembly of FIG. 7A.

FIG. 8A is a perspective view of another example of a needle guard assembly, according to an aspect of the present disclosure.

FIG. 8B is a perspective view of a cross-section of the needle guard assembly of FIG. 8A.

FIG. 8C is an exploded cross-sectional view of the needle guard assembly of FIG. 8A.

FIG. 9A is a perspective view of another example of a needle guard assembly, according to an aspect of the present disclosure.

FIG. 9B is an exploded perspective view of the needle guard assembly of FIG. 9A.

FIGS. 10A-10E are cross-sectional views of the needle guard assembly of FIG. 9A showing the needle cannula being inserted into and removed from the cover member, according to aspects of the present disclosure.

FIG. 11A is a perspective view of another example of a needle guard assembly connected to a fluid delivery device, according to an aspect of the present disclosure.

FIG. 11B is a perspective view of the needle guard assembly of FIG. 11A.

FIG. 11C is an exploded perspective view of the needle guard assembly of FIG. 11A.

FIG. 12A is a front view of the needle guard assembly and fluid delivery device of FIG. 11A in a pre-use position with the cover member over the distal end of the needle cannula, according to an aspect of the present disclosure.

FIG. 12B is a front view of the needle guard assembly and fluid delivery device of FIG. 11A with the needle guard assembly inserted into an ampoule for aspirating a medical solution from an interior of the ampoule into a syringe barrel of the fluid delivery device.

FIG. 12C is a front view of the needle guard assembly and fluid delivery device of FIG. 12C, with the cover member in a retracted position.

FIG. 12D is a front view of the needle guard assembly and fluid delivery device of FIG. 12C, with the cover member in a retracted position after expelling the medical solution from the syringe barrel.

FIG. 12E is a front view of the needle guard assembly and fluid delivery device of FIG. 11A in a post-use position after the medical solution is expelled from the syringe barrel and the cover member returns to an extended position over the distal end of the needle cannula.

FIG. 13A is a perspective view of a portion of the needle guard assembly of FIG. 11A with the needle guard assembly in a pre-use extended position, according to an aspect of the present disclosure.

FIG. 13B is a perspective view of a portion of the needle guard assembly of FIG. 11A with the needle guard assembly in a retracted position.

FIG. 13C is a perspective view of a portion of the needle guard assembly of FIG. 11A with the needle guard assembly in a post-use extended position, with the cover member over the distal end of the needle cannula.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. As used herein, the term “proximal” refers to a portion or end of a device, such as a syringe or catheter, which is grasped, manipulated, or used by a practitioner or another user. The term “distal” refers to an end or portion of the device that is farthest away from the portion of the device that is grasped, manipulated, or used by the practitioner. For example, the “proximal end” of a catheter or IV line refers to the end including a fluid port that is connected to a fluid container, such as an IV bag or syringe. The “distal end” of the catheter or IV line refers to the end that is connected to the patient. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

With reference to the figures, the present disclosure is directed to needle guard assemblies 10 and fluid delivery devices, such as syringes 110, configured to be used by medical practitioners, such as clinicians or healthcare workers, for performing fluid delivery or infusion procedures for patients. As used herein, the “healthcare worker” can be a medical professional, such as a medical technician or nurse, trained to perform a medical procedure, such as a fluid delivery or blood collection procedure. The present disclosure is also directed to drug delivery and infusion methods performed by the practitioners using the needle guard assemblies 10 and syringes 110 disclosed herein. In some examples, the medical solution delivered to the patient can be a medication, a total parenteral nutrient (TPN) liquid, or another therapeutic agent used for treatment of chronic or acute conditions. Exemplary therapeutic agents can include, for example, drugs, chemicals, biological or biochemical substances that, when delivered in a therapeutically effective amount to the patient, achieve a desired therapeutic effect.

The assemblies 10 and syringes 110 disclosed herein are configured so that the medical solution contained in the ampoule 112 can be aspirated into a barrel of the syringe 110 and injected from the syringe barrel to the patient through the same injection (e.g., hypodermic) needle. Accordingly, the needle guard assemblies 10, syringes 110, and methods disclosed herein do not require use of both a filter needle and a hypodermic needle to perform a fluid injection procedure. As such, problems of procedures that use multiple needles, such as heightened contamination risk, more opportunities for needle sticks, and time delays, can be avoided.

In some examples, the needle guard assemblies 10 disclosed herein include liquid-permeable structures or portions for filtering glass shards from the medical solution before the medical solution is drawn into the injection needle. For example, the liquid-permeable structures or portions can include filters, such as filters formed from permeable sheets or films, as well as members comprising perforations, holes, openings, slots, slits, and other structures for permitting liquid flow towards the injection needle, while preventing solid particles, such as glass shards, from being drawn into the injection needle.

In some examples, the needle guard assemblies 10 can also include elastomeric seals within the needle guard or shield for creating a sealed cavity, space, or region around a distal tip of the needle. The elastomeric seal can be provided to ensure that sufficient suction is created around the distal tip of the needle for aspirating the medical solution from the ampoule 112 into the needle. The elastomeric seals can comprise a thermoplastic elastomer, such as but not limited to a styrenic block copolymer, a thermoplastic vulcanizate, such as ethylene propylene diene monomer rubber (EPDM) rubber, or a cross-linked rubber, such as polyisoprene or butyl rubber

### Needle Guard Assemblies Including a Separate Needle Shield and Ampoule Shield

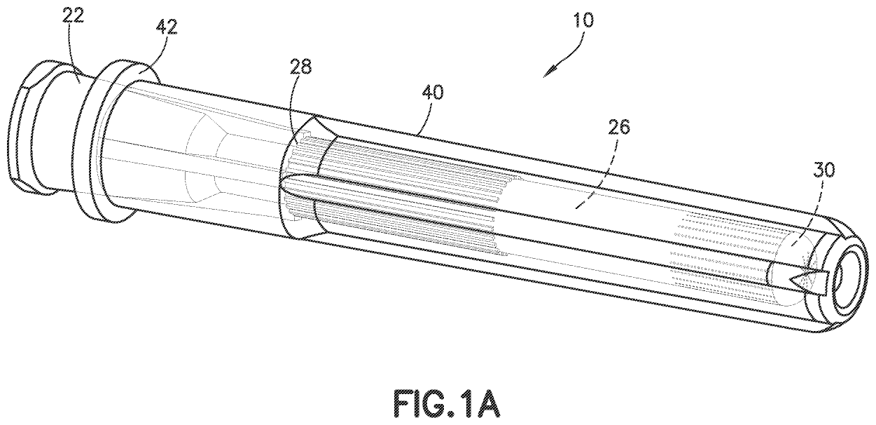

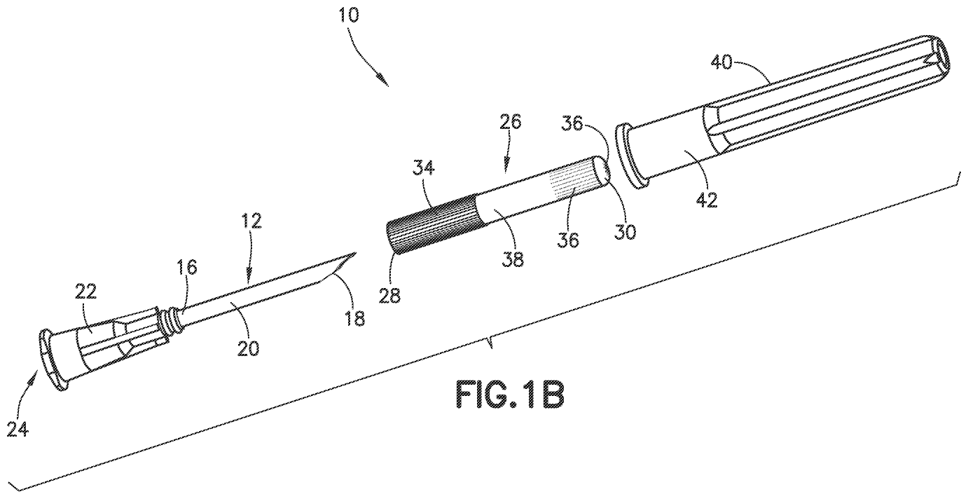

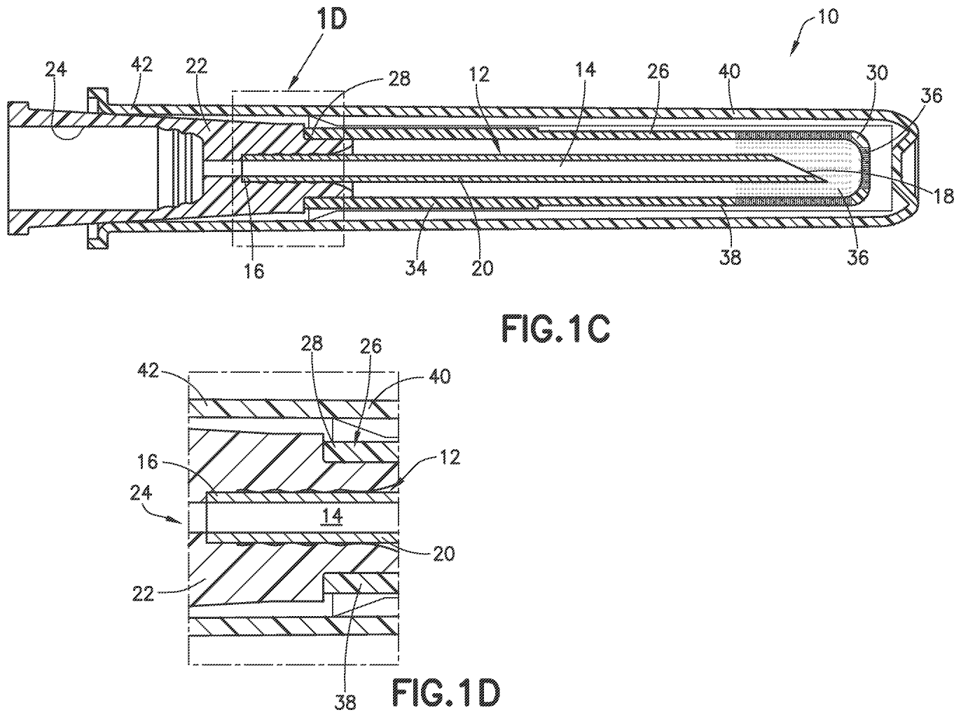

FIGS. 1A-1D show a needle guard assembly 10 configured to be mounted to a fluid delivery device, such as a syringe 110, for aspirating fluid, such as a medical solution, into a barrel of the syringe 110 and for expelling the medical solution from the barrel to a patient through the needle guard assembly 10. Specifically, FIG. 1A is a perspective view of the needle guard assembly 10 showing exterior features of the assembly 10. FIG. 1B is an exploded perspective view of the assembly 10. FIGS. 1C and 1D are cross-sectional views of the assembly 10.

As shown in FIGS. 1A-1D, the assembly 10 comprises a needle cannula 12 defining a lumen 14 having a proximal end 16, a distal end 18 with a sharpened tip, and a tubular body 20 extending between the proximal end 16 and the distal end 18. The needle cannula 12 can be a cannula of a conventional hypodermic needle, as are known in the art, such as an injection needle configured for subcutaneous injection. The needle cannula 12 can also be an injection needle used for intramuscular and/or intravenous injections, as are known in the art. The needle cannula 12 can be formed from various rigid biocompatible materials, such as metal (e.g., stainless steel) or rigid plastics. For example, the needle cannula 12 can comprise a rigid thermoplastic polymer or a bioabsorbable rigid polymer.

The needle guard assembly 10 further comprises a needle hub 22 connected to the proximal end 16 of the needle cannula 12. The needle hub 22 is configured to be mounted to the syringe 110 to provide fluid communication between the syringe 110 and the lumen 14 of the needle cannula 12 through a central passageway 24 of the needle hub 22. For example, as shown most clearly in FIG. 1C, the needle hub 22 can comprise a cavity, such as a female luer cavity, including a tapered sidewall (e.g., a sidewall having a taper of about 6%) sized to receive a stem of a male luer connector extending from a distal end of the syringe 110. The central passageway 24 extends axially through the needle hub 22 from a distal end of the cavity to a distal end of the needle hub 22. As shown in FIG. 1C, the proximal end 16 of the needle cannula 12 is received within the passageway 24.

In some examples, the needle hub 22 is formed from a resilient, deformable, and/or high friction material for enhancing a connection between the syringe 110, the needle cannula 12, and the needle hub 22. For example, the needle hub 22 can be a molded part formed from rigid thermoplastic, such as at least one of polycarbonate, high density polyethylene, polypropylene, polyether ketone (PEEK), polytetrafluoroethylene (PTFE), acrylonitrile butadiene styrene (ABS), biaxially-oriented polyethylene terephthalate (BOPET), and/or polyimide. Friction between surfaces of the cavity of the needle hub 22 and the distal end of the syringe 110 can be sufficient to provide a removable, but secure and fluid-tight connection and seal between the syringe 110 and the needle hub 22. In a similar manner, the proximal end 16 of the needle cannula 22 can be received within the channel or passageway 24 of the hub 22. Friction between the outer surface of the tubular body 20 of the needle cannula 12 and an inner surface of the channel or passageway 24 can be sufficient to retain the needle cannula 12 within the needle hub 22.

The needle guard assembly 10 can further comprise a liquid-permeable ampoule shield or cover member 26 extending over at least the distal end 18 of the needle cannula 12. As used herein, a “liquid-permeable” member or material is a member or material that is sufficiently porous and/or includes a sufficient number of perforations, holes, slots, or openings to permit a substantial volume of liquid to pass through a portion of the member or material. The “liquid-permeable” member or material can be configured to prevent solid particles from passing through the member or material. For example, the “liquid-permeable” member or material can be a filter, porous sponge, mesh, woven structure, or structure comprising holes, openings, or perforations of sufficient size to prevent the solid particles from passing through the member or material. In some examples, the liquid-permeable cover member 26 is configured to filter solid particles, such as glass shards, from a medical solution before the medical solution is drawn into the lumen 14 of the needle cannula 12.

In some examples, the ampoule shield or cover member 26 can be a substantially rigid structure defining a cylindrical cavity enclosing the sharpened tip at the distal end 18 of the needle cannula 12. For example, the cover member 26 can be formed from a rigid thermoplastic, such as at least one of polycarbonate, high density polyethylene (HDPE), polypropylene (PP), polyether ketone (PEEK), polytetrafluoroethylene (PTFE), acrylonitrile butadiene styrene (ABS), biaxially-oriented polyethylene terephthalate (BOPET), and/or polyimide. Further, the cover member 26 can be sized so that all portions of the needle cannula 12 are spaced apart from the cover member 26, meaning that no portion of the needle cannula 12 contacts an inner surface of the cover member 26.

In some examples, the cover member 26 can be a generally tubular structure comprising a proximal end 28 engaged to the needle hub 22 and a closed distal end 30 enclosing the distal end 18 of the needle cannula 12. For example, the proximal end 28 of the cover member 26 can be configured to slide over and frictionally engage an outer surface of the needle hub 22 for retaining the cover member 26 on the needle hub 22. More specifically, as shown in FIGS. 1C and 1D, the needle hub 22 can include a cylindrical distal portion comprising an annular outer surface. The proximal end 28 of the cover member 26 is sized to be inserted over the annular outer surface, thereby forming the friction engagement between the inner surface of the cover member 26 and the annular surface of the needle hub 22. As previously described, the needle hub 22 can be formed from the resilient, deformable, and/or high-friction material, which can enhance the connection between the cover member 26 and the needle hub 22.

In some examples, the connection or interface between the inner surface of the cover member 26 and the needle hub 22 is an airtight and liquid-tight seal preventing air or liquids from entering the space defined by the cover member 26 through the open proximal end 28 of the cover member 26. By sealing the proximal end 28 of the cover member 26, a substantial suction force can be created in the space or cavity around the distal end 18 of the needle cannula 12 as the medical solution is aspirated into the needle cannula 12. The created suction force is sufficient to draw the medical solution through the liquid-permeable portion(s) of the cover member 26 and into the lumen 14 of the needle cannula 12.

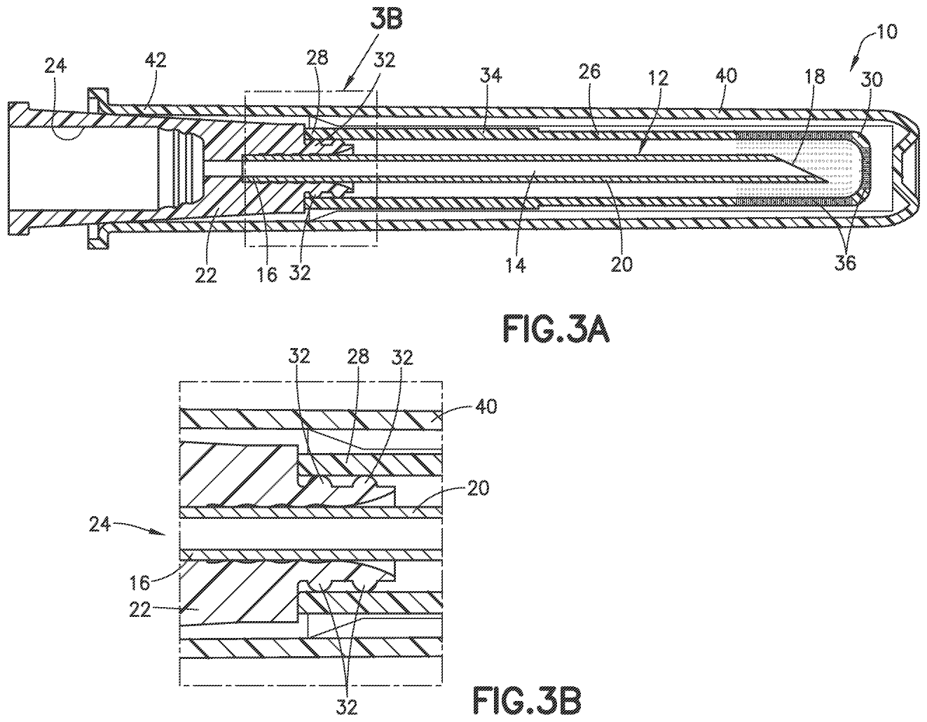

FIGS. 3A and 3B show another exemplary needle guard assembly 10 including the needle cannula 12, the needle hub 22, and the ampoule shield or cover member 26. However, the exemplary needle guard assembly 10 of FIGS. 3A and 3B differs from the previous examples because protrusions, ridges, or ribs are added to the outer surface of the distal end of the needle hub 22 for enhancing the connection between the inner surface of the cover member 26 and the modified needle hub 22. More specifically, as shown in FIGS. 3A and 3B, the distal portion of the needle hub 22 includes two annular ridges 32 extending radially outward from the outer surface of the needle hub 22. The annular ridges 32 are in contact with the inner surface of the cover member 26 for forming a secure and air-tight seal for the cover member 26.

In some examples, the ampoule shield or cover member 26 is a molded part including various molded features for improving usability and/or maneuverability of the cover member 26. As described in further detail herein, the practitioner may be required to grasp the cover member 26 (e.g., between an index finger and thumb of one hand) in order to remove the cover member 26 from the needle hub 22 exposing the needle cannula 12 enclosed within the cover member 26. In order to improve maneuverability, the cover member 26 can include a grip portion 34. For example, as shown in FIG. 1B, the grip portion 34 can comprise axially extending raised ridges molded on the outer surface of the cover member 26.

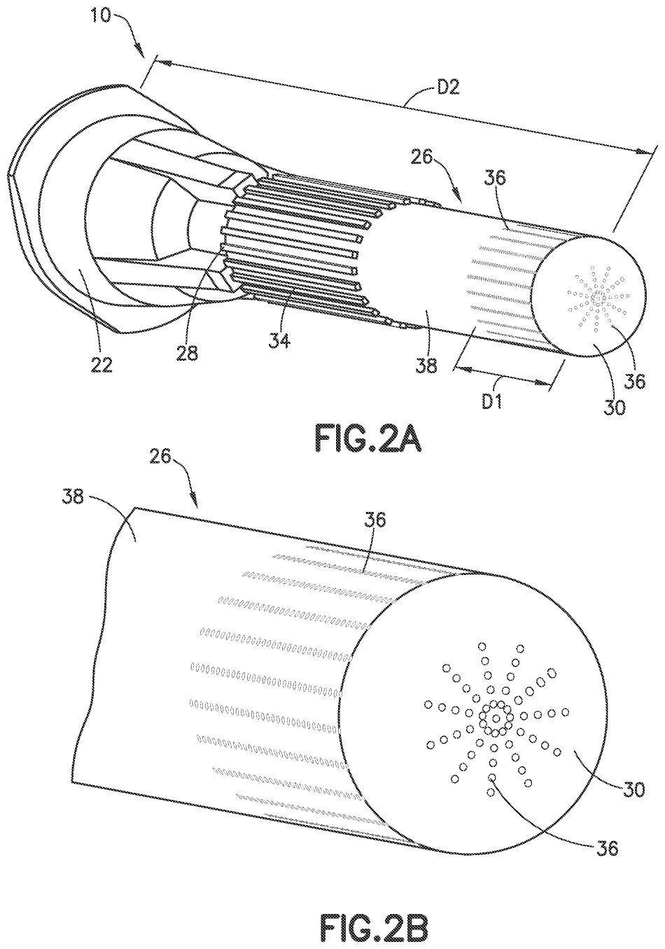

In some examples, as shown in FIGS. 1A-1D, as well as FIGS. 2A and 2B, the cover member 26 also includes perforations 36, holes, openings, or other indentations formed on portions of a sidewall 38 and/or closed distal end 30 of the cover member 26 for permitting liquid, such as medical solution contained in the ampoule 112, to pass through the cover member 26 and toward the distal end 18 of the needle cannula 12. The perforations 36 can comprise holes that are less than or equal to about 5 microns in diameter, or preferably from about 1 micron to about 5 microns in diameter. The perforations 36 can be through-holes extending through the sidewall 38 and/or closed distal end 30 of the cover member 26 made by laser cutting, heat staking, or similar micromachining and/or cutting techniques.

The size of the perforations 36 (less than or approximately equal to 5 microns) is selected to limit larger particles, such as glass shards, from passing into the needle cannula 12. The number and size of perforations 36 should be sufficient to allow aspiration of fluid through the cover member 26 without creating significant resistance to the fluid aspiration. For example, an 18G needle with an internal diameter of 0.838 mm can be used as a filter needle. Assuming that the filter in the commercial 18G filter needle creates an order of magnitude increase in back pressure, the number of perforations 36 or holes can be calculated to be approximately 2,800 (e.g., internal surface area of 18G filter/(surface area of one hole×10)). The perforation 36 or holes can be arranged in any pattern including linear, hexagonal, spiral, or others. In some examples, a hexagonal arrangement is preferred, which provides a maximum density of perforations 36 concentrated near the tip of the needle cannula 12.

An exemplary perforation pattern that can be provided on the cover member 26 of the present disclosure is shown most clearly in FIGS. 2A and 2B. As shown in FIGS. 2A and 2B, the perforation pattern can include a first group of perforations 36 extending through the sidewall 38 of the cover member 26. The first group of perforations 36 are arranged to form axial lines extending parallel to a longitudinal axis of the cover member 36. The axial lines generally extend from a distal-most portion of the sidewall 38 towards a middle portion of the sidewall 38. For example, the axial lines of perforations 36 can extend axially from the distal-most portion of the sidewall 38 by a distance DI of from about 27 mm to about 30 mm in length, which, as shown in FIG. 2A, is about 20% to about 40% of a total axial length D2 of the cover member 26. For example, the total length D2 of the cover member 26 can be from about 7 cm to about 11 cm.

In other examples, the axial lines may extend from the distal-most portion of the sidewall 38 to the middle of the cover member 26, beyond the middle of the cover member 26, or to any other convenient position determined, for example, based upon a desired flow rate or flow volume for liquid passing through the cover member 26. In some specific examples, as shown in FIG. 2B, the axial lines can include about thirty individual perforations 36. Further, as shown in FIGS. 2A and 2B, there are axial lines of perforations 36 around the entire circumference of the sidewall 38, meaning that the medical solution can be drawn into the cover member 26 from any direction. In other examples, the axial lines may only be present on some portions, quadrants, or sections of the sidewall 38, while other portions, sections, quadrants, or areas of the sidewall 38 can be free from perforations 36.

The second group of perforations 36 are positioned on the closed distal end 30 of the cover member 26. For example, the cover member 26 can include perforations 36 arranged in radial lines extending from a common point. Specifically, as shown in FIG. 2B, the cover member 26 includes eleven radial lines of perforations 36 extending from a central point of the closed distal end 30 of the cover member 26. Each radial line includes four equidistantly spaced perforations 36. In other examples, the perforation pattern on the closed distal end 30 can include fewer or more than eleven radial lines of perforations 36. Further, each radial line can include fewer or more than four perforations depending, for example, upon the desired flow rate or flow volume for medical solution flowing into the cover member 26. Also, in other examples, perforations 36 on the distal end 30 of the cover member 26 can be provided in a variety of other patterns, such as spirals, square-shaped groups, rectangle-shaped groups, groups of parallel lines of perforations 36, or other convenient patterns that can be easily made by laser cutting, heat skating, or over convenient micromachining processes.

In some examples, the needle guard assembly 10 further comprises exterior packaging, such as a needle shield or outer shield 40, configured to be positioned over the cover member 26 for protecting the cover member 26 and needle cannula 12 prior to use. The outer shield 40 can be tubular packaging placed over the cover member 26 during manufacture or transport to protect the cover member 26 from any pre-use damage, such as damage that occurs during shipping or while the needle guard assembly 10 is in storage at a medical facility prior to use. The outer shield 40 can be configured to prevent dirt, dust, debris, liquids, solutions, and other contaminants from contacting surfaces of the cover member 26 and/or from passing through the perforations 36 of the cover member 26 and contaminating the needle cannula 12.

In some examples, the outer shield 40 can comprise a proximal end or proximal portion 42 configured to engage a portion of the outer surface of the needle hub 22 for securing the outer shield 40 to the needle hub 22, as shown most clearly in FIG. 1C. For example, as shown in FIG. 1C, the proximal portion 42 of the outer shield 40 can be in contact with the outer surface of the needle hub 22 at a position proximal to the area of contact between the cover member 26 and the needle hub 22. The outer shield 40 can be a substantially rigid structure defining a cavity or space sized to receive the cover member 26 and the needle cannula 12 enclosed therein. For example, the outer shield 40 can be formed from the same rigid thermoplastic material as the cover member 26 or from a different type of thermoplastic material, as are known in the art. In particular, the outer shield 40 can be formed from one or more of polycarbonate, high density polyethylene (HDPE), polypropylene, polyether ketone (PEEK), polytetrafluoroethylene (PTFE), acrylonitrile butadiene styrene (ABS), biaxially-oriented polyethylene terephthalate (BOPET), and/or polyimide. Also, the outer shield 40 can be shaped so that it does not contact any portion of the cover member 26, meaning that there is a gap between portions of the outer surface of the cover member 26 and the inner surface of the outer shield 40. Accordingly, the outer shield 40 does not press against, scratch, abrade, or otherwise deform any portions of the cover member 26 as the outer shield 40 is being inserted on or removed from the needle hub 22.

### Fluid Delivery Device for use With the Needle Guard Assembly

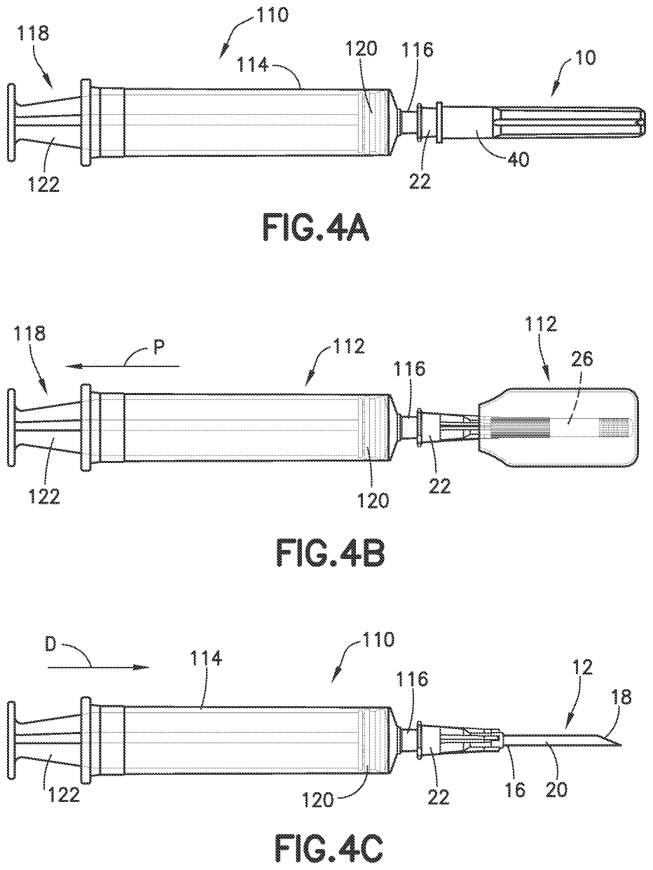

As previously described, the needle guard assemblies 10 of the present disclosure are configured to be mounted to a distal end of a fluid delivery device, such as the syringe 110. Exemplary syringes 110 that can be used with the needle guard assemblies 10 disclosed herein are shown, for example, in FIGS. 4A-4C.

As shown in FIGS. 4A-4C, the syringe 110 comprises a barrel 114 comprising a distal tip 116 defining a channel for expelling fluid, such as a medical solution, from an interior of the syringe barrel 114. The distal tip 116 can be a tapered stem or male luer connector configured to be inserted into and/or engage a corresponding female needless or luer connector. As used herein, a “luer connector” refers to a connector that includes a tapered portion (i.e., a luer taper) for creating a friction engagement between the distal tip 116 and a tapered cavity, such as a cavity of a female luer connector, configured to receive and engage the distal tip 116. More specifically, in some examples, the distal tip 116 can be configured to be used with female needleless connectors, specifically female luer connectors for a range of dimensions permitted by various design protocols, such as ISO 80369-7:2016 or ISO 80369-7:2021 (Female Luer Lock Connector dimensions).

The syringe 110 can further comprise a plunger 118 positioned in the syringe barrel 114 for expelling the medical solution from the syringe barrel 114 through the channel of the distal tip 116. In some examples, the plunger 118 comprises a stopper 120 and a plunger rod 122 connected to and extending from the stopper 120 through an open proximal end of the syringe barrel 114. As with conventional stoppers known in the art, an outer peripheral surface of the stopper 120 can be configured to seal against an inner surface of the sidewall of the syringe barrel 114 for moving the medical solution through the syringe barrel 114. For example, the practitioner can grasp the plunger rod 122 for moving the stopper 120 in a proximal direction to aspirate fluid into the syringe barrel 114 through the distal tip 116 and for moving the stopper 120 through the syringe barrel 114 in the distal direction to expel the medical solution from the syringe barrel 114 through the distal tip 116.

### Method for Aspirating Medical Fluid From a Glass Ampoule

As previously described, the needle guard assembly 10 disclosed herein is used by the practitioner to aspirate a medical solution from a glass ampoule or vial 112 into the syringe barrel 114 of the syringe 110 and to inject the medical solution from the syringe barrel 114 to the patient through the same injection needle (e.g., a hypodermic needle) used for aspiration of the medical solution into the syringe barrel 114. Steps for using the needle guard assembly 10 and syringe 110 connected thereto for aspiration of a medical solution and for injection of the medical solution to the patient are illustrated in FIGS. 4A-4C.

Initially, the practitioner obtains the needle guard assembly 10 and syringe 110. The practitioner may also obtain the glass ampoule 112 containing the medical solution to be injected to the patient. In order to prepare the medical devices and other tools for use, the practitioner can remove any packaging from the needle guard assembly 10, syringe 110, and ampoule 112. For example, the practitioner can remove a tip cap or shield from the distal tip 116 of the syringe 110, thereby exposing the distal tip 116 of the syringe 110 so that it can be connected to other devices or tools. The practitioner can also remove any caps or covers engaged to the plunger rod 122 for holding the plunger rod 122 in place preventing the stopper 120 from moving through the syringe barrel 114 at inappropriate or unexpected times. Once the packaging is removed, the practitioner attaches the needle guard assembly 10 to the needle hub 22 by, for example, inserting the distal tip 116, such as the male needleless connector or male luer connector, of the syringe 110 into the tapered cavity of the needle hub 22, thereby securing the syringe 110 to the needle hub 22 of the needle guard assembly 10 in a removable, but sealed fluid-tight connection. The needle guard assembly 10 is shown attached to the syringe 110 in a pre-use or ready-for-use position in FIG. 4A. The practitioner can also remove any packaging, such as a cap, cover, protective wrapping, or other protective materials from the ampoule 112. Shortly before beginning to perform a fluid delivery procedure, the practitioner can also break the narrow neck of the ampoule 112 in order to gain access the medical solution contained in the ampoule 112.

Once the needle guard assembly 10 is mounted to the syringe 10, the practitioner removes the outer shield 40 from the needle guard assembly 10. The practitioner next advances the stopper 120 through the syringe barrel 114 by a small amount in a distal direction to remove air from the interior of the syringe barrel 114 by, for example, pressing on a thumb plate of the plunger rod 122 while grasping a finger flange of the syringe barrel 114 between, for example, a ring finger and an index finger. Once any air is removed from the interior of the syringe barrel 114, the practitioner next inserts the needle guard assembly 10, including the cover member 26 and needle cannula 12, into the ampoule 112 through the open end created by breaking the neck portion of the ampoule 112, which places the needle cannula 12 of the needle guard assembly 10 in fluid communication with the medical solution contained in the ampoule 112. Once the needle cannula 12 is positioned in the ampoule 112, the practitioner retracts the stopper 120 of the syringe 10 by, for example, pulling the thumb press plate of the plunger rod 122 away from the syringe barrel 114, thereby moving the stopper 120 in a proximal direction (shown by arrow P in FIG. 4B) through the syringe barrel 114. Movement of the stopper 120 in the proximal direction draws the medical solution through the perforations 36 of the cover member 26 and into the lumen 14 of the needle cannula 12. However, any solid particles, such as glass shards created by breaking the neck of the glass ampoule 112, do not pass through the cover member 26 and are not drawn into the needle cannula 12.

Once a sufficient amount of the medical solution is drawn into the syringe barrel 114, the practitioner removes the needle guard assembly 10 from the interior of the ampoule 112. The practitioner then removes the ampoule shield or cover member 26 from the needle guard assembly 10 by, for example, grasping the grip portion 34 of the cover member 26 between the thumb and index finger, and pulling the cover member 26 axially away from the needle hub 22 to expose the sharpened tip on the distal end 18 of the needle cannula 12.

The practitioner can then perform the injection by inserting the sharpened tip of the needle cannula 12 into the skin, muscle tissue, and/or vasculature of the patient. Once the needle cannula 12 is in place, the practitioner can then move the stopper 120 in the distal direction (shown by arrow D in FIG. 4C) through the interior of the syringe barrel 114 using the plunger rod 122 to expel the medical solution from the syringe barrel 114 and through the distal tip 116 into the lumen 14 of the needle cannula 12. For example, as previously described, the practitioner can press the thumb press plate of the plunger rod 122 with the thumb while grasping the finger flange of the syringe barrel 114 to move the plunger rod 122 in the distal direction (shown by arrow D in FIG. 4C), which causes the stopper 120 to move distally through the syringe barrel 114 to force the medical solution from the syringe barrel 114. The practitioner continues to move the stopper 120 distally through the syringe barrel 114 until all or substantially all of the medical solution is expelled from the syringe barrel 114 through the distal tip 116 and into the needle cannula 12. The syringe 10 and needle cannula 12 attached thereto are shown in the final or end-of-use position in FIG. 4C.

### Needle Guard Assembly With Internal Filter

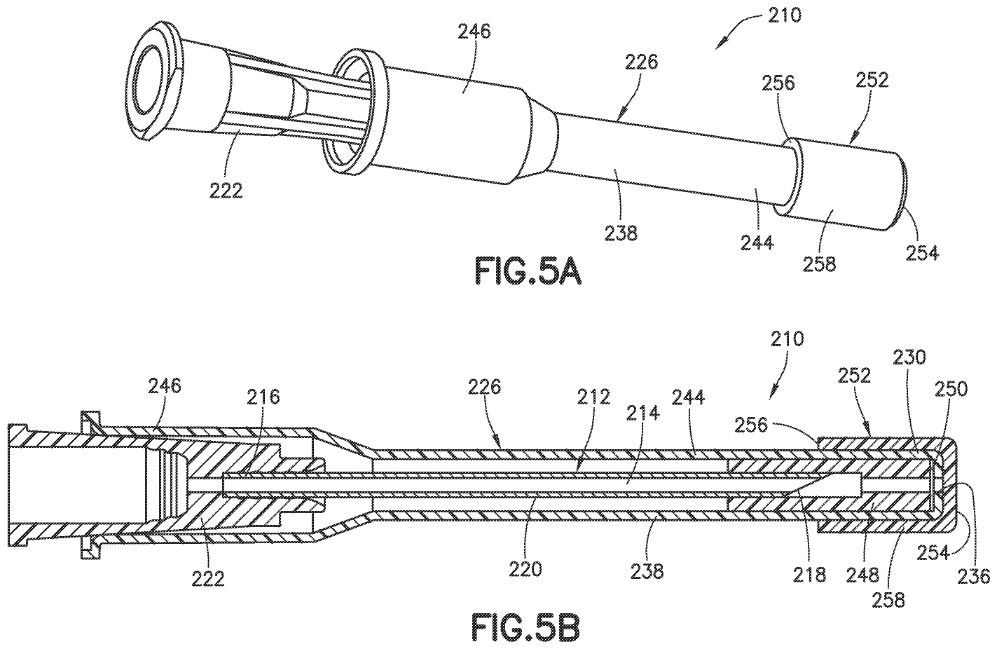

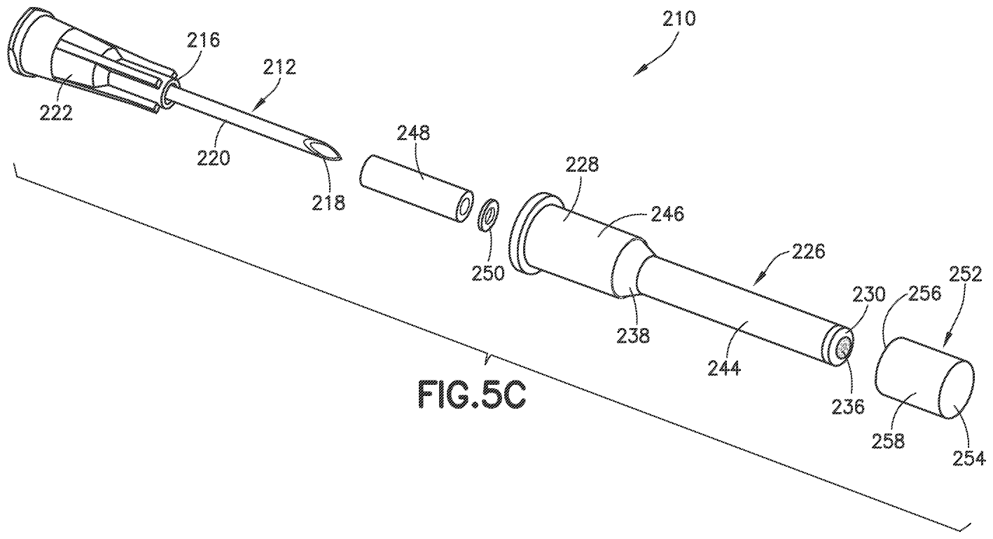

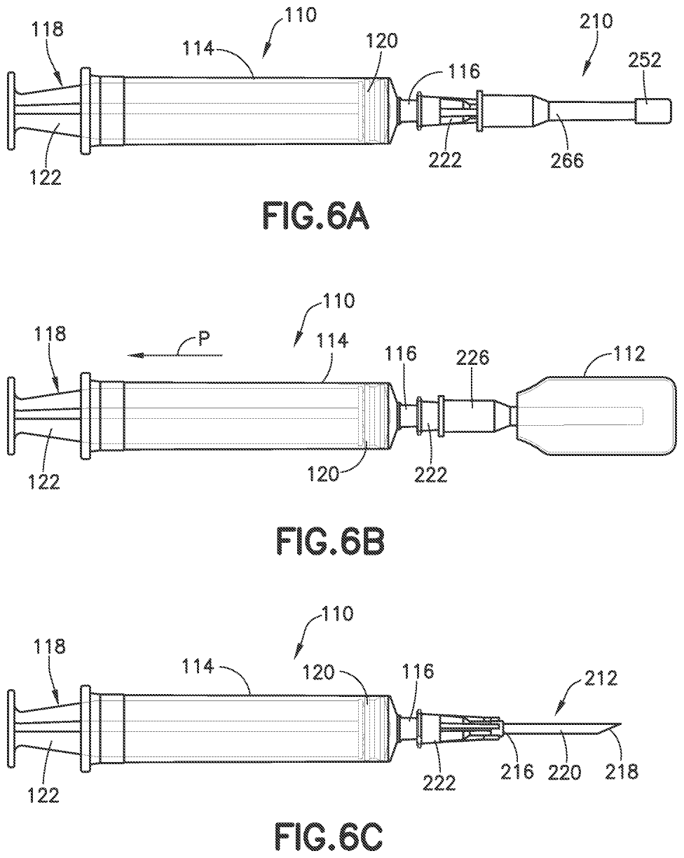

FIGS. 5A-6C show another exemplary needle guard assembly 210 configured to be connected to the syringe 110 for aspirating the medical solution from the ampoule 112 to the syringe barrel 114, while separating solid particles, such as glass shards, from the medical solution. As in previous examples, the needle guard assembly 210 comprises the needle cannula 212 defining the lumen 214 comprising the proximal end 216, a distal end 218 including the sharpened tip, and a tubular body 220 extending therebetween. The needle guard assembly 210 further comprises the needle hub 222 connected to the proximal end 216 of the needle cannula 212 configured to be mounted to the distal tip 116 of the syringe 110. The needle guard assembly 210 also includes a liquid-permeable cover member 226.

However, unlike in previous examples, the liquid-permeable cover member 226 of FIGS. 5A-6C forms both external packaging for the needle guard assembly 210 for protecting the needle cannula 212 during, for example, manufacture and transport, and also the filter or shield that prevents solid particles, such as glass shards, from contacting the needle cannula 212 enclosed by the cover member 226. More specifically, as shown in FIGS. 5A-5C, the cover member 226 can be a tiered tubular structure including a narrow distal tier 244 over the distal end 218 and sharpened tip of the needle cannula 212 and a wider proximal tier 246 enclosing the proximal end 216 of the needle cannula 212 and portions of the needle hub 222. As shown in FIG. 5B, an inner surface of the proximal tier 246 contacts and forms a frictional engagement with a portion of the needle hub 222. The cover member 226 also includes one or more holes, openings, or perforations 236 extending through a closed distal end 230 of the cover member 226 for permitting the medical solution to pass through the cover member 226 to the needle cannula 212 during aspiration of the medical solution to the needle cannula 212.

With continued reference to FIGS. 5A-5C, the needle guard assembly 210 further comprises a cylindrical support 248 or seal positioned at the distal end 230 of the cover member 226 for protecting and/or supporting the needle cannula 212. As shown in FIGS. 5B and 5C, the cylindrical support 248 or seal can include a central channel comprising a proximal portion sized to receive the distal end 218 of the needle cannula 212 and a narrowed distal portion extending through the cylindrical support 248. The central channel can be axially aligned with one or more of the holes, openings, or perforations 236 extending through the closed distal end 230 of the cover member 226.

The needle assembly 210 also includes a filter 250 positioned between the cylindrical support 248 and the closed distal end 230 of the cover member 226. The filter 250 is positioned so that the medical solution aspirated through the cover member 226 passes through the filter 250 and then into the lumen 214 of the needle cannula 212. In some examples, the filter 250 is a flat sheet, such as a sheet of filter paper, having an outer diameter matching an inner diameter of the distal end 230 or distal tier 244 of the cover member 226. For example, the filter 250 can comprise a sheet or film designed to retain solid particles, such as glass shards, as well as other contaminants. In particular, the filter 250 can be configured to retain particles having a minimum size of at least 5 microns. In some examples, the filter 250 is a Versapor® membrane (e.g., Versapor® 5000 membrane disc), which is a woven acrylic membrane disc, manufactured by Pall Corporation.

In some examples, the needle guard assembly 210 also includes a removable cap 252 configured to be positioned over the distal end 230 and/or distal tier 244 of the cover member 226. For example, the cap 252 can be a cylindrical structure comprising a closed distal end 254 sized to receive the closed distal end 230 of the cover member 226, an outer proximal end 256, and a sidewall 258 extending between the distal end 254 and the proximal end 256. The sidewall 258 of the cap 252 can be configured to contact an outer surface of the cover member 226 forming a friction engagement with the cover member 226 for retaining the cap 252 on the cover member 226. As with the previously described outer shield 40 (shown in FIGS. 1A-1D), the cap 252 can be applied to the cover member 226 during manufacture and transport to protect the cover member 226 and to prevent contamination of the needle cannula 212. When the needle guard assembly 210 is ready for use, the practitioner can remove the cap 252 from the cover member 226 prior to inserting the needle guard assembly 210 into the ampoule 112.

FIGS. 6A-6C are front views of the needle guard assembly 210 and syringe 110 showing steps for aspiration of the medical solution into the syringe barrel 114 and for expelling the medical solution from the syringe barrel 114 during a fluid injection procedure. The syringe 110 and the needle guard assembly 10 are shown in an initial or pre-use position in FIG. 6A. As shown in FIG. 6A, the needle guard assembly 10 is mounted to the distal tip 116 of the syringe 110. The cap 252 is in place over the distal end 230 or distal tier 244 of the cover member 226. Also, the stopper 120 is in a distal-most position seated against a distal end of the syringe barrel 114.

When ready for use, the practitioner removes the cap 252 from the cover member 226 and, as shown in FIG. 6B, inserts the needle guard assembly 210 into the ampoule 112. In order to aspirate the medical solution from the interior of the ampoule 112 to the syringe barrel 114, the practitioner pulls the plunger rod 122 in the proximal direction (shown by arrow P in FIG. 6B), which moves the stopper 120 distally in order to draw the medical solution through the one or more holes, openings, or perforations 236 on the closed distal end 230 of the cover member 226. The medical solution then moves through the filter 250, where solid particles (e.g., solid particles that are at least 5 microns in size) are retained by the filter 250, and into the central channel of the cylindrical support 248 or seal. Specifically, the filer 250 is configured to retain solid particles, such as glass shards, which prevents the solid particles from being drawn into the channel of the cylindrical support 248 or seal along with the medical solution. Continued proximal movement of stopper 120 draws the medical solution from the channel into the lumen 214 of the needle cannula 212 and from the lumen 214 to the syringe barrel 114. As in previous examples, after a sufficient volume of medical solution is drawn into the syringe barrel 114, the practitioner can remove the needle guard assembly 210 from the interior of the ampoule 112. The practitioner then removes the cover member 226 from the needle cannula 212 exposing the sharpened tip of the needle cannula 212, as shown in FIG. 6C. The practitioner can then perform the injection, as previously described, by inserting the sharpened tip of the needle cannula 212 into the skin, muscle tissue, or vasculature of the patient and moving the stopper 120 in the distal direction through the syringe barrel 114 expelling the medical solution from the syringe barrel 114 and through the lumen 214 of the needle cannula 212.

### Needle Guard Assemblies With Internal Sealing Rings

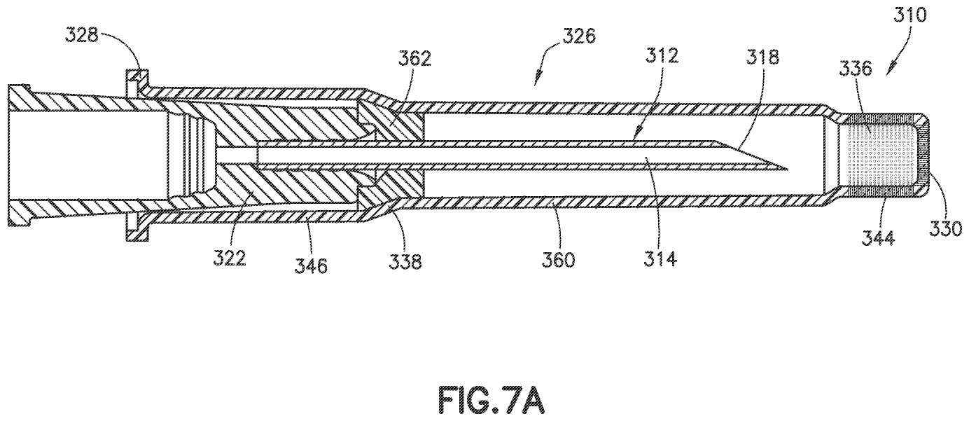

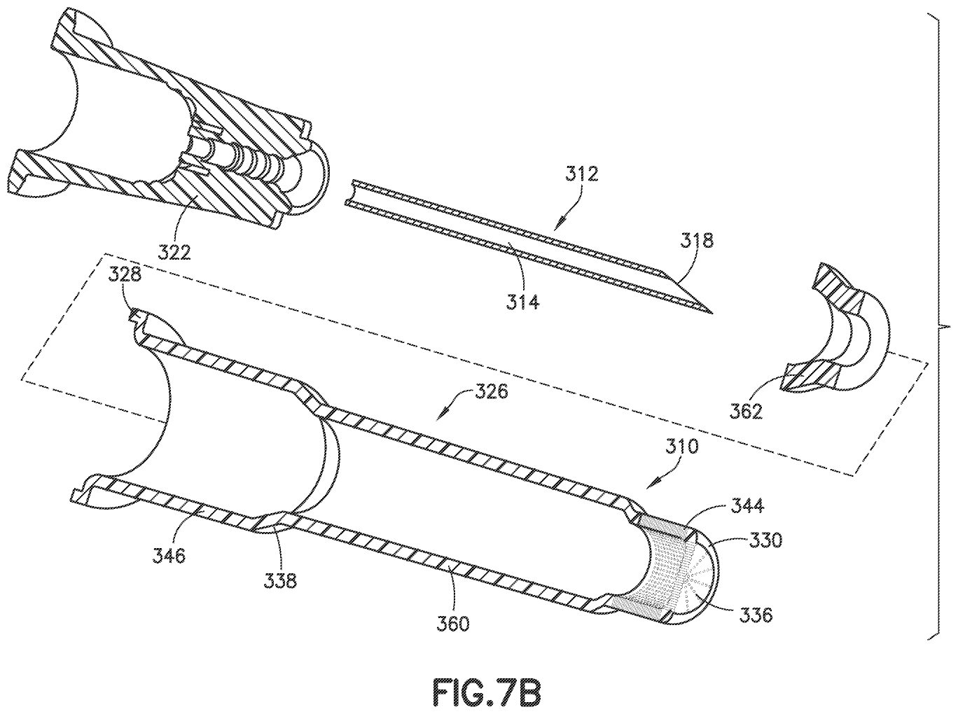

FIGS. 7A-10E show additional examples of needle guard assemblies 310 including either stationary or movable internal sealing rings disposed about the needle cannula 312 and enclosed within the cover member 326. As described in further detail herein, the sealing rings are configured to create a suction space or suction cavity within the cover member 326 to improve aspiration of the medical solution through the cover member 326 and into the lumen 314 of the needle cannula 312.

As in previous examples, the needle guard assembly 310 includes the needle cannula 312 defining a lumen 314 comprising the distal end 318 with the sharpened tip and the needle hub 322 for securing the needle cannula 312 to the distal tip 116 of the syringe 110. The needle guard assembly 310 also includes the liquid-permeable cover member 326 extending over the needle cannula 312. The cover member 326 includes a proximal end 328 engaged to the needle hub 322 and a closed distal end 330 enclosing the sharpened tip of the needle cannula 312. As in previous examples, the cover member 310 is configured to be exterior packaging for the cover member 326 and needle cannula 312, as well as a filter or shield for blocking solid particles, such as glass shards, from being drawn into the lumen 314 of the needle cannula 312. The needle guard assemblies 310 shown in FIGS. 7A-10E differ from previous examples and from each other in the structure and position of the sealing rings disposed about the needle cannula 312. Also, in some examples, the sealing rings are stationary fixed to the inner surface of the cover member 326. In other examples, as described herein, the sealing rings can be moveable through the interior of the cover member 326.

As shown in FIGS. 7A and 7B, the needle guard assembly 310 can comprise a tiered cover member 326 including a narrow distal tier 344, an intermediate tier 360, and a wider proximal tier 346. As in previous examples, an inner surface of the proximal tier 346 is frictionally engaged to an outer surface of the needle hub 322, thereby securing the cover member 326 to the needle hub 322. As shown in FIGS. 7A and 7B, the cover member 326 includes multiple perforations 336 or openings on the distal tier 344 of the cover member 326. For example, there can be perforations 336 or openings extending through a sidewall 338 of the cover member 326. There can also be perforations 336 or openings through the closed distal end 330 of the cover member 326. For example, as in previous examples, the perforations 336 or openings extending through the sidewall 338 of the cover member 326 can be arranged in axial lines, while perforations 336 on the closed distal end 330 can be arranged in radially extending lines extending from a common or central point.

The needle guard assembly 310 also includes the sealing ring 362 which, as shown in FIGS. 7A and 7B, is a tapered annular structure including a central opening sized to receive the needle cannula 312. The sealing ring 362 can be an elastomeric structure (e.g., a structure formed from a thermoplastic elastomer) configured to form a seal between the outer surface of the needle cannula 312 and the inner surface of the cover member 326. As shown in FIG. 7A, the sealing ring 362 is seated in the interior of the cover member 326 between the proximal tier 346 and the intermediate tier 360 of the cover member 326. Further, the sealing ring 362 is configured to be immovably retained within the cover member 326 meaning that the needle cannula 312 slides through the sealing ring 362 while inserting the needle cannula 312 into the cover member 326 or removing the cover member 326 from the needle cannula 312. However, the sealing ring 362 remains fixed within the cover member 326 at the position between the proximal tier 346 and the intermediate tier 360.

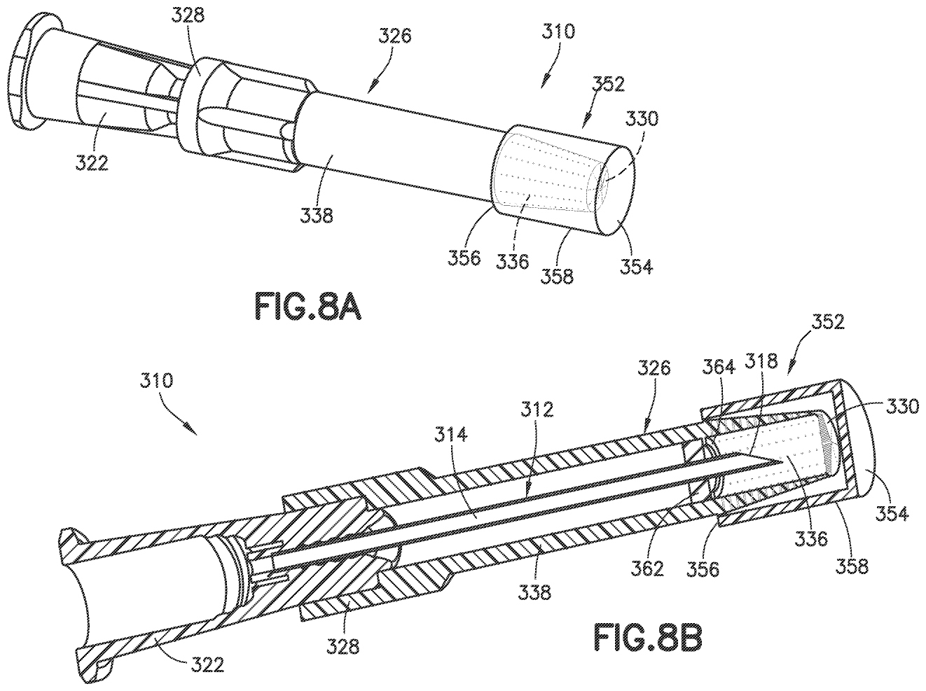

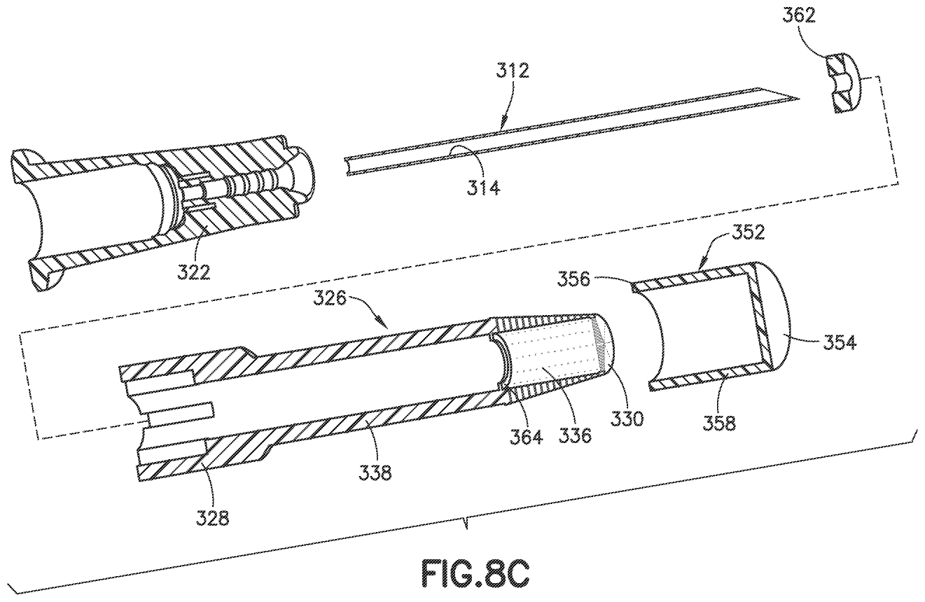

FIGS. 8A-8C show another example of the needle guard assembly 310 including an immovable sealing ring 362. More specifically, as shown in FIGS. 8A-8C, the needle guard assembly 310 includes the needle cannula 312, the needle hub 322, and the cover member 326 comprising the distal end 330 with the perforations 336, as in previous examples. The cover member 326 of FIGS. 8A-8C comprises the proximal end 328 engaged to the needle hub 322 and the closed distal end 330 enclosing the distal end 318 and the sharpened tip of the needle cannula 312. Unlike in previous examples, the distal end 330 of the cover member 326 is tapered providing a frusto-conical shaped distal end 330. Also, unlike in previous examples, the cover member 326 comprises an annular shelf 364 extending radially inwardly from the inner surface of the sidewall 338 of the cover member 326. For example, as shown in FIG. 8B, the shelf 364 is positioned adjacent to proximal-most perforations 336 of the cover member 326.

The needle guard assembly 310 also includes the sealing ring 362. Unlike in previous examples, the sealing ring 362 of FIGS. 8A and 8B is a disc shaped structure including a flat proximal surface and an opposing flat distal surface. As shown in FIG. 8B, the sealing ring 362 is seated on the shelf 364, with the distal surface of the sealing ring 362 in contact with a proximal facing surface of the shelf 364. The sealing ring 362 is intended to be an immovable sealing structure that remains in place seated against the shelf 364 during use of the needle guard assembly 310 and syringe 110. In particular, the needle cannula 312 is sized to slide through the central opening of the sealing ring 362 as the cover member 326 is inserted over the needle cannula 312 or removed from the needle cannula 312. Also, as previously described, the sealing ring 362 creates the sealed cavity or space about the distal end 318 of the needle cannula 312 that allows for creation of a suction force sufficient to draw the medical solution through the perforations 336 of the cover member 326 and into the lumen 314 of the needle cannula 312.

As in previous examples, the needle guard assembly 310 can also include the removable cap 352 sized to fit over and engage the distal end 330 of the cover member 326 for sealing and/or preventing contamination of the cover member 326 or needle cannula 312 until the practitioner is ready to perform the fluid aspiration and injection procedure. As in previous examples, the cap 352 comprises the closed distal end 354, the open proximal end 356, and the sidewall 358 extending therebetween. As in previous examples, the practitioner removes the cap 352 prior to inserting the needle guard assembly 310 into the interior of the ampoule 112.

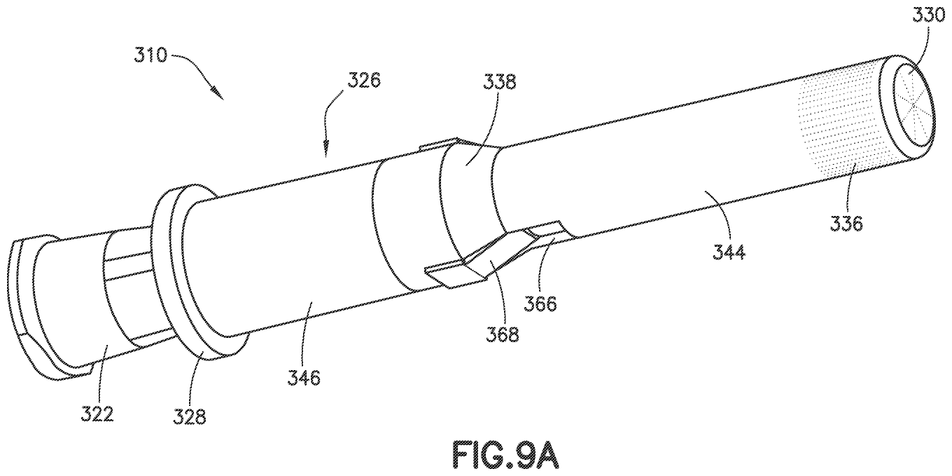

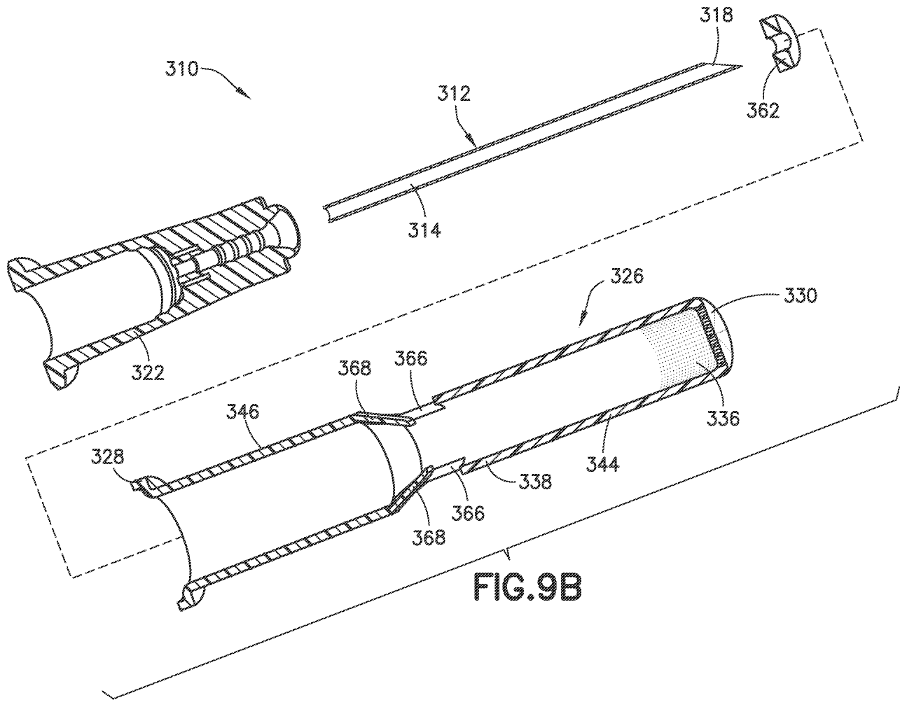

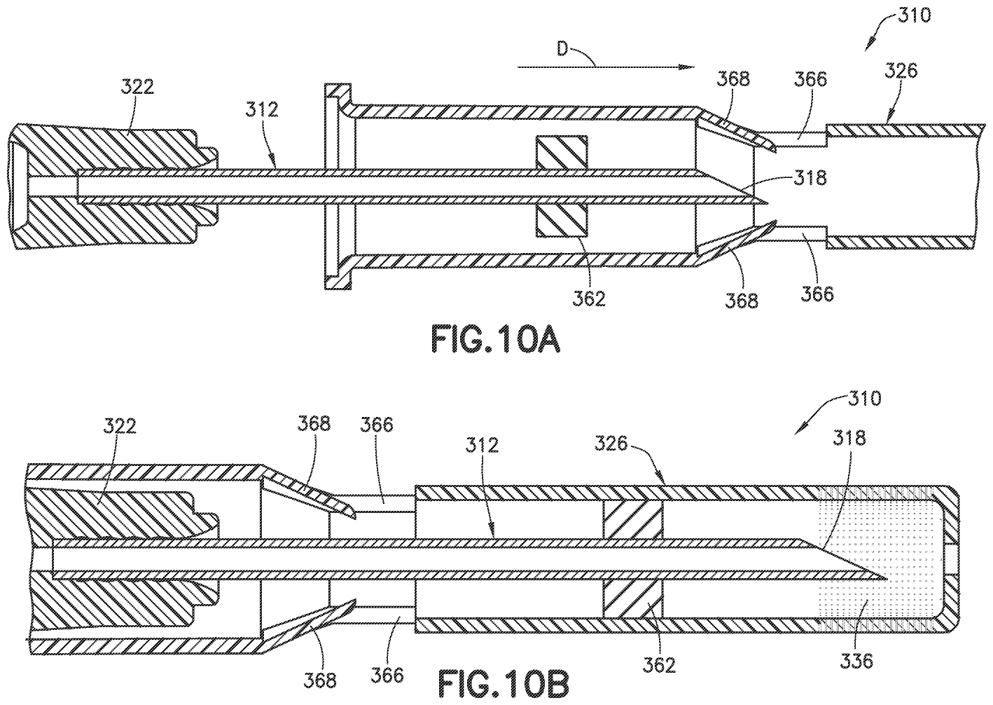

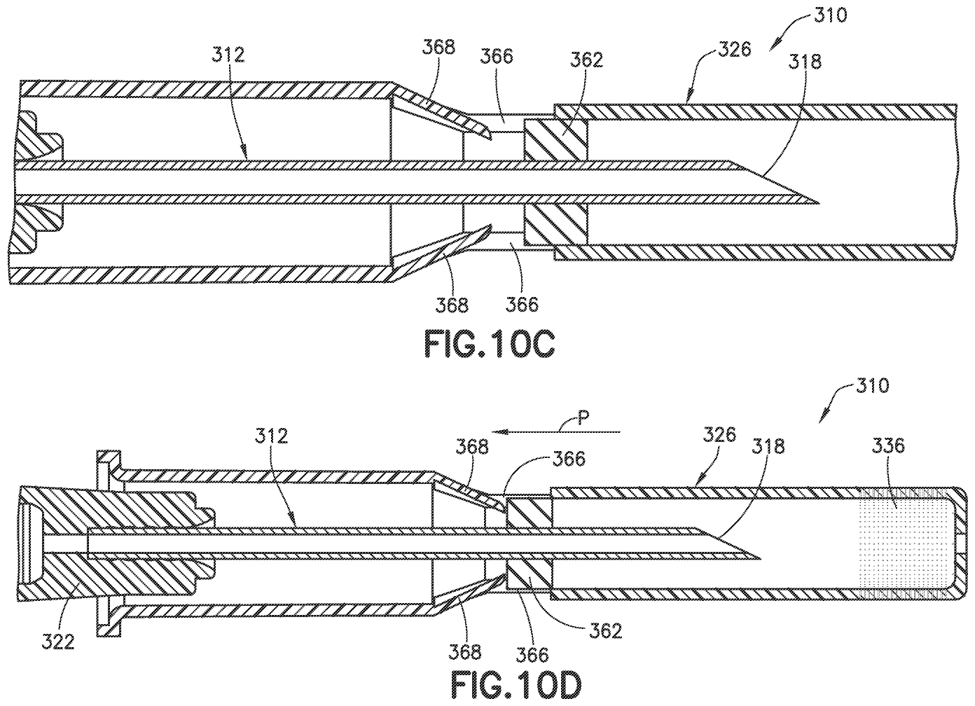

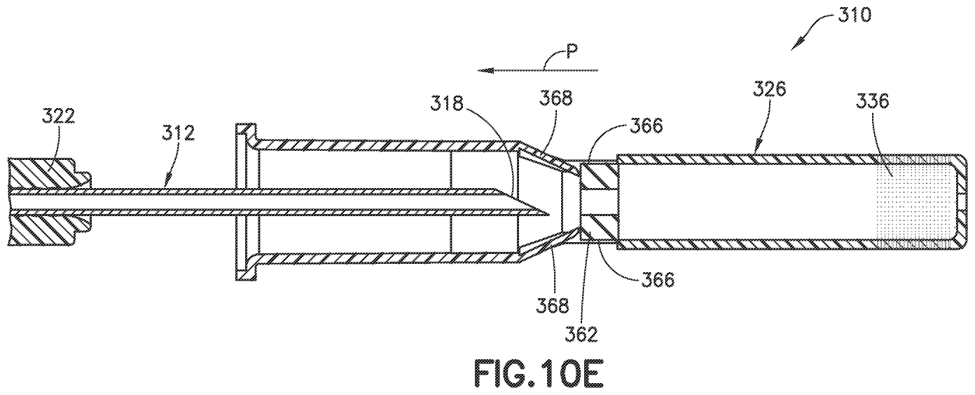

FIGS. 9A-10E show another example of a needle guard assembly 310, which includes a movable sealing ring 362. The moveable sealing ring 362 can be configured to move through the interior of the cover member 326 as the cover member 326 is inserted over or removed from the needle cannula 312. As shown in FIGS. 9A-10E, the needle guard assembly 310 includes the needle cannula 312 defining the lumen 314, the needle hub 322, and the cover member 326. As in previous examples, the cover member 326 of FIGS. 9A-10E is a tiered structure including the distal tier 344 comprising the perforations 336 for drawing the medical solution towards the lumen 314 of the needle cannula 312, and the proximal tier 346 configured to frictionally engage the outer surface of the needle hub 322. Unlike in previous examples, however, the cover member 326 of FIGS. 9A-10E also includes retaining structures, such as slots, tabs, detents, protrusions, or similar structures for removing the sealing ring 362 from the needle cannula 312 and for retaining the sealing ring 362 within the cover member 326 as the needle cannula 312 is removed from the cover member 326.

More specifically, as shown in FIGS. 9B and 10A-10E, the sealing ring 362 is a disk-shaped structure including the flat proximal surface and the opposing flat distal surface. The sealing ring 362 also includes the central bore or channel sized to receive and frictionally engage the outer surface of the needle cannula 312. The cover member 326 includes an arrangement of slots 366 and/or inwardly angled protrusions, surfaces, or tabs 368 for retaining the sealing ring 362 within the cover member 326. In particular, the sealing ring 362 can be sized to be received within the slots 366 and/or to contact the tabs 368 of the cover member 326 for separating the sealing ring 362 from the needle cannula 312 and for preventing the sealing ring 362 from being removed from the cover member 326. The slots 366 and/or tabs 368 can be arranged so that the scaling ring 362 moves past the slots 366 and/or tabs 368 as the needle cannula 312 is being inserted into the cover member 326. However, the slots 366 and/or tabs 368 can be configured to engage the sealing ring 362 as the needle cannula 312 is being removed from the cover member 326, thereby preventing the sealing ring 362 from being removed from the cover member 326 along with the needle cannula 312.

FIGS. 10A-10E are cross-sectional views showing steps for insertion and removal of the needle cannula 312 from the cover member 326 of the needle guard assembly 310. Specifically, as shown in FIG. 10A, the needle cannula 312 and sealing ring 362 attached thereto are inserted into the cover member 326. Specifically, the practitioner moves the needle cannula 312 in a distal direction (shown by arrow D in FIG. 10A) through the cover member 326. During the distal movement of the needle cannula 326, the sealing ring 362 moves past the tabs 368 of the cover member 326 causing the tabs 368 to deflect radially outward away from the sealing ring 362, which allows the needle cannula 312 and the sealing ring 362 to continue to move distally through the interior of the cover member 326 to a distal position. The needle cannula 312 is shown in the distal position in FIG. 10B with the distal end 318 and sharpened tip of the needle cannula 312 positioned proximate to the perforations 336 on the distal end 330 of the cover member 326.

As previously described, after the medical solution is drawn into the barrel 114 of the syringe 110, the practitioner can begin to remove the cover member 326 from the needle cannula 312 to expose the sharpened tip at the distal end 318 of the needle cannula 312 for performing the injection to the patient. As shown in FIG. 10C, the needle cannula 312 is being withdrawn from the cover member 326, which moves the sealing ring 362 proximally through the interior of the cover member 326 toward the slots 366 and the tabs 368. FIG. 10D shows the scaling ring 362 positioned in the slot 366 and retained in place by the tabs 368. Once the scaling ring 362 is retained within the slots 366 and in contact with the tabs 368, the needle cannula 312 can be separated from the sealing ring 362 and removed from the cover member 326 by continuing to move the needle cannula 312 in the proximal direction (shown by arrow P in FIGS. 10D and 10E). While the needle cannula 312 is moved in the proximal direction, the sealing ring 362 is retained in the cover member 326 by the slots 366 and tabs 368, thereby separating the needle cannula 312 from the sealing ring 362. As shown in FIG. 10E, the needle cannula 312 is shown separated from the sealing ring 362 after being removed from the proximal end 328 of the cover member 326 and ready for use to perform a fluid injection to the patient.

### Needle Guard Assembly With Support Arms

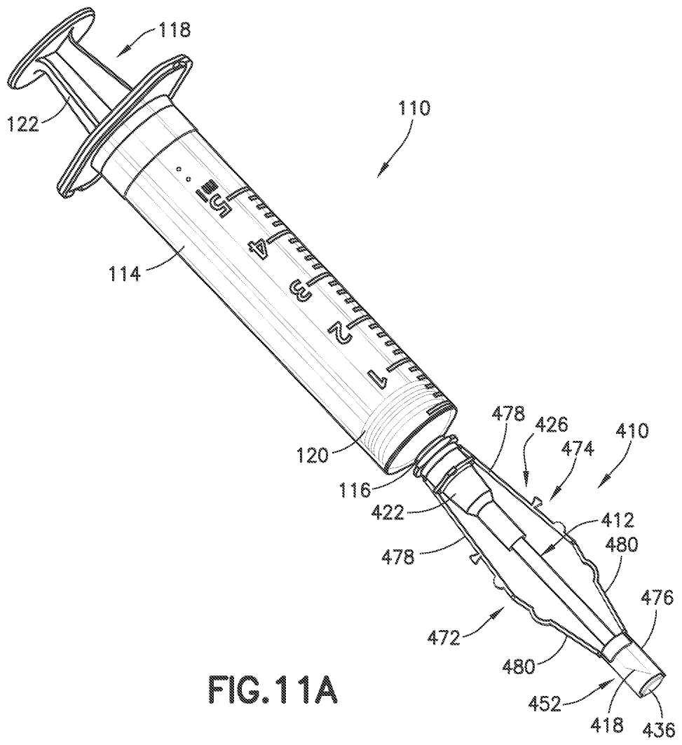

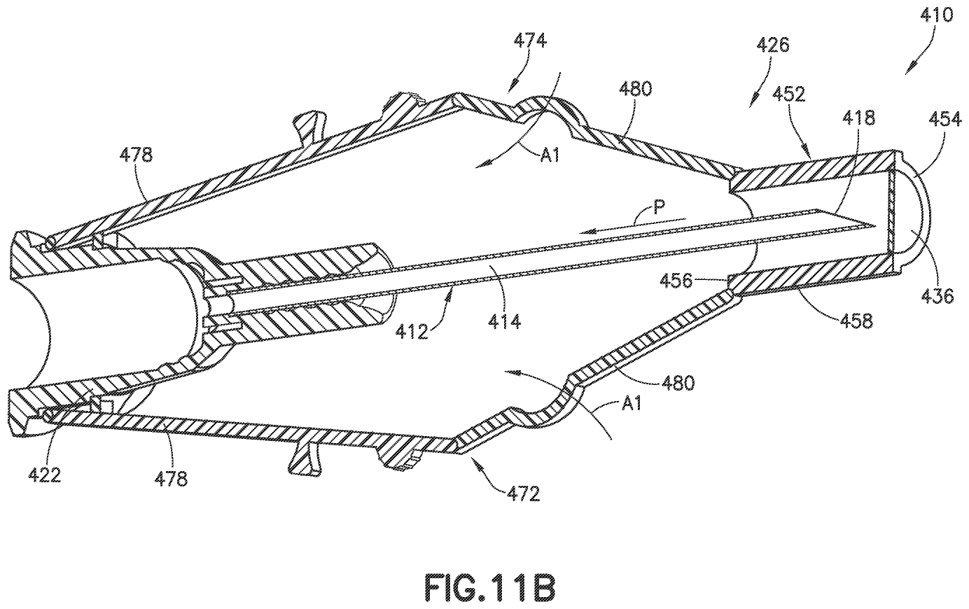

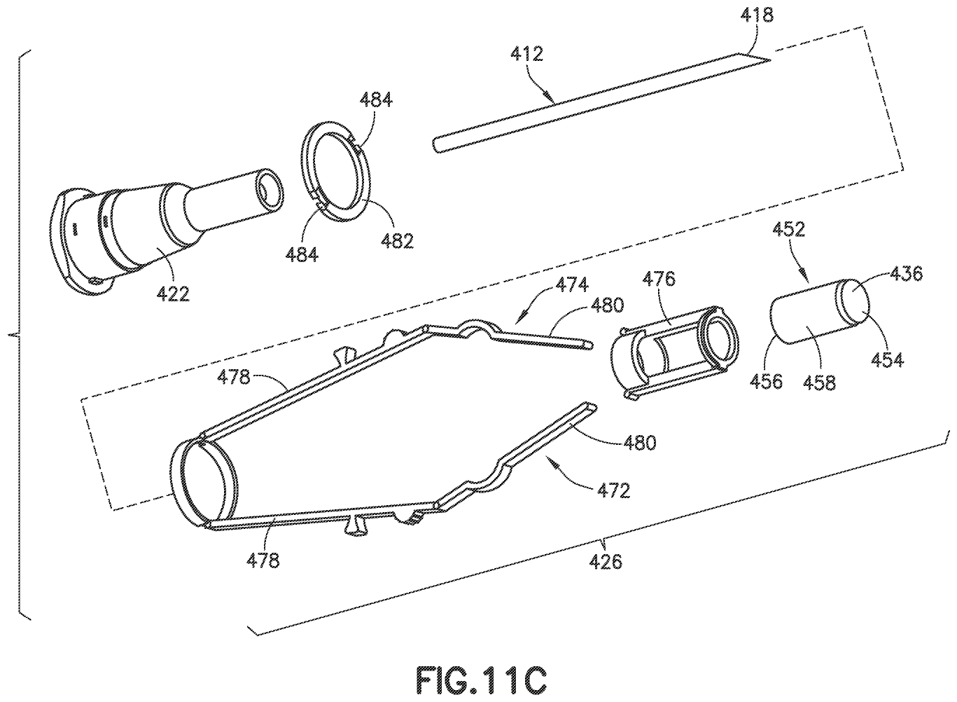

FIGS. 11A-13C show another example of a needle guard assembly 410, in which the elongated tubular body that forms the cover member of previous examples is replaced by a cover member 426 comprising a smaller cap 452 or enclosure around the distal end 418 of the needle cannula 412 and elongated members or arms 472, 474 for moving the cap 452 relative to the needle cannula 412.

More specifically, as shown in FIGS. 11A-11C, the needle guard assembly 410 comprises the needle cannula 412 defining the lumen 414 comprising the distal end 418 with the sharpened tip and needle hub 422 of previous examples. As in previous examples, the needle cannula 412 can be an injection needle (e.g., a hypodermic needle) for subcutaneous, intra-muscular, and/or vascular injection of the medical solution to a patient. As in previous examples, the needle cannula 412 is used both for aspirating the medical solution from the vial or ampoule 112 into the syringe barrel 114 and for injecting the medical solution from the syringe barrel 114 to the patient through the lumen 414 of the needle cannula 412.

However, unlike in previous examples, the cover member 426 extending over the distal end of the needle cannula 412 comprises the cap 452, which comprises an open proximal end 456, a closed distal end 454, and a cylindrical sidewall 458 extending therebetween positioned over the distal end 418 of the needle cannula 412. The cap 452 can be formed from a rigid thermoplastic material including perforations 436, openings, holes, slots, and similar structures that permit the medical solution to pass through the cap 452, while preventing solid particles, such as shards of glass, from passing through the cap 452. In other examples, the cap 452 can comprise woven filters, meshes, or similar materials for preventing solid particles from passing through the cap 452 toward the lumen 414 of the needle cannula 412. In some examples, the cap 452 can be enclosed or supported by a frame or receptacle 476, such as a basket formed from elongated tines, for supporting the cap 452 and for protecting the needle cannula 412 enclosed by the cap 452.

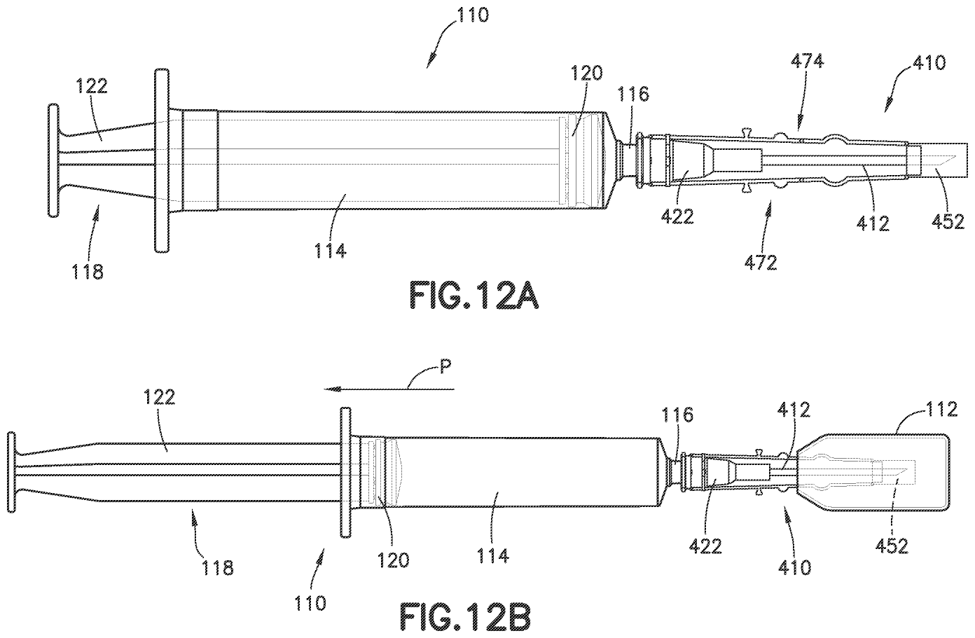

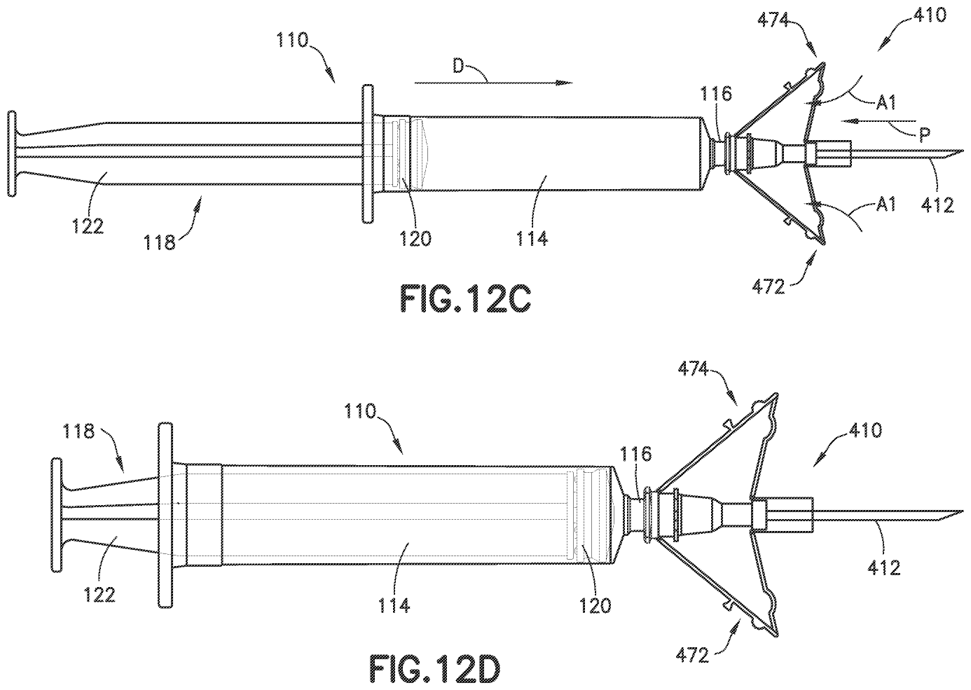

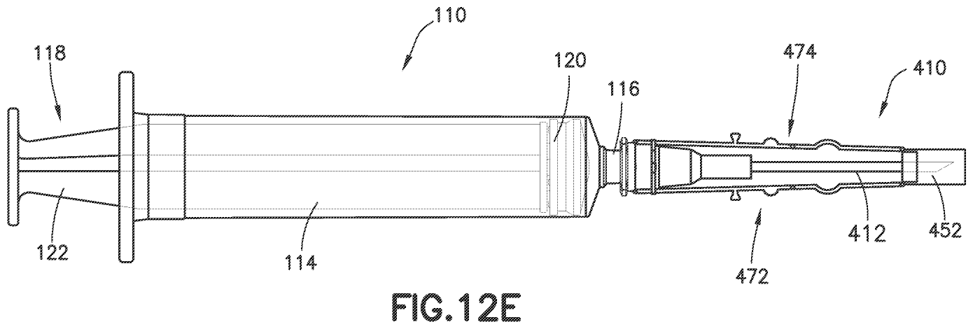

As described in further detail herein, the closed distal end 454 of the cap 452 can also comprise a pierceable material that can be punctured by the sharpened tip of the needle cannula 412 as the cap 452 and arms 472, 474 move from an initial position (shown in FIGS. 11A and 12A) to a retracted position (shown in FIGS. 12C and 12D).

The cover member 426 further comprises the articulating or bending arms 472, 474 for supporting the receptacle 476 and the cap 452 and for moving the cap 452 axially away from the distal end 418 of the needle cannula 412 so that the needle cannula 412 can be used for injection of the medical solution to the patient. For example, the cover member 426 can include a slider arrangement or mechanism comprising a first arm 472 and a second arm 474 mounted between the needle hub 422 and the cap 452 for moving the cap 452 along the needle cannula 412 between the initial position (shown in FIGS. 11A and 12A), where the cap 452 covers the distal end 418 of the needle cannula 412, and the retracted position (shown in FIGS. 12C and 12D), where the cap 452 is positioned on a sidewall or tubular body of the needle cannula 412 at a proximal position. As shown in FIGS. 12B and 12C, in the retracted position, the cap 452 can be retained near the proximal end 416 of the needle cannula 412 proximate to the needle hub 422.

In some examples, the first arm 472 and/or the second arm 474 are formed from or comprise a proximal section or segment 478 hinged or flexibly connected to a distal section or segment 480. The distal section or segment 480 can be configured to bend toward the proximal section or segment 478, as shown by arrows A1 (shown in FIGS. 11B and 12C). Bending of the distal segments 480 toward the proximal segments 478 causes portions of the arms 472, 474 to move radially outward away from the needle cannula 412, which causes the cap 452 to move in a proximal direction (shown by arrow P in FIGS. 11B and 12C) from the distal end 418 of the needle cannula 412 toward to needle hub 422.

Movement of the arms 472, 474 toward the retracted position causes the closed distal end 454 of the cap 452 to contact the sharpened tip of the needle cannula 412, which pierces the closed distal end 454 of the cap 452. Continued proximal movement of the arms 472, 474 moves the pierced cap 452 along the needle cannula 412 to the retracted position. As shown in FIG. 12C, when fully retracted, the cap 452 is over the proximal end 416 of the needle cannula 412 proximate to the needle hub 422.

In some examples, the first arm 472 and the second arm 474 can be biased to a particular position meaning that the arms 472, 474 automatically move toward the biased position once any retaining structures, clips, or hooks are removed or disengaged from the arms 472, 474. For example, the first arm 472 and the second arm 474 can be biased to the retracted position. In such cases, the arms 472, 474 may automatically move toward the retracted position without pressing or otherwise contacting the arms 472, 474 and may remain in the retracted position until the practitioner presses against the arms 472, 474 with sufficient force to overcome the biasing force and to begin to move the arms 472, 474 back towards the initial or extended position.

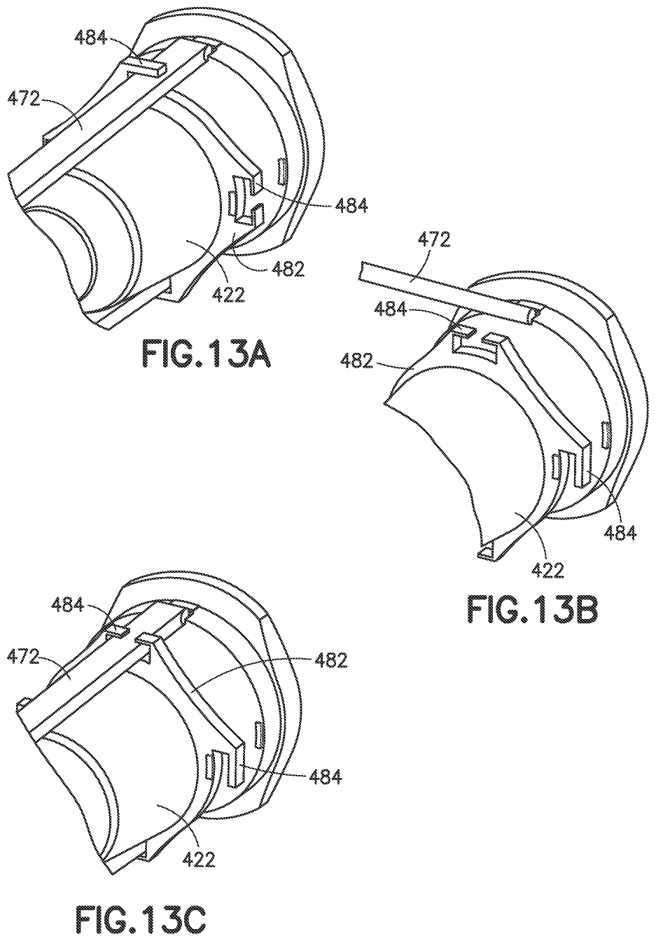

In some examples, as shown in FIGS. 13A-13C, the needle guard assembly 410 also includes a retention ring 482 positioned about the needle hub 422 configured to engage a portion of the first arm 472 and/or the second arm 474 for retaining the cap 452 over the distal end 418 of the needle cannula 412. When the first arm 472 and/or the second arm 474 are released from the retention ring 482, the arms 472, 474 can be configured to move from the extended or initial position to the retracted position.

In some examples, the retention ring 482 can be an elastomeric and/or deformable structure including deformable or bendable hooks 484 configured to engage the first arm 472 and/or the second arm 474 for maintaining the arms 472, 474 in the extended or initial positon. For example, as shown in FIG. 13A, prior to use, the arms 472, 474 are in the initial or extended position. One or both of the arms 472, 474 are retained by the hooks 484 of the retention ring 482, which prevents the arms 472, 474 from moving to the retracted position until released by the practitioner. As shown in FIG. 13B, once the arms 472, 474 are released from the hooks 482, the arms 472, 474 move from the extended or initial position to the retracted position. In some examples, after injection of the medical solution to the patient through the needle cannula 412, the practitioner can allow the needle guard assembly 410 to return to the extended or initial position. For example, the practitioner can press the arms 472, 474 radially inwardly causing at least one of the arms 472, 474 to be received within the hooks 484, as shown in FIG. 13C. Once received within the hooks 484, the arms 472, 474 are retained in the extended or initial position protecting the practitioner from inadvertently contacting the sharpened tip on the distal end 418 of the needle cannula 412.

FIGS. 12A-12E are front views of the syringe 110 and the needle guard assembly 410 showing movement of the arms 472, 474 of the needle guard assembly 410 during the fluid injection procedure. Initially, as shown in FIG. 12A, the needle guard assembly 410 is attached to the syringe 110 with the cap 452 positioned over the sharpened tip at the distal end 418 of the needle cannula 412. In this initial position, the arms 472, 474 are extended in order to hold the cap 452 in a desired position over the distal end 418 of the needle cannula 412. As previously described, the arms 472, 474 can be maintained in the extended or initial position by the hooks 484 of the retention ring 482, as shown in FIG. 13A.

When ready for use, the practitioner inserts the needle guard assembly 410 including cap 452 and the needle cannula 412 into the ampoule 112 containing the medical solution. The practitioner then moves the stopper 120 of the syringe 110 in a proximal direction (shown by arrow P in FIG. 12B) using the plunger rod 122 to draw the medical solution from the ampoule 112 through the cap 452 and into the lumen 414 of the needle cannula 412. As in previous examples, the cap 452 is configured to permit the medical solution to pass through the cap 452 and to the lumen 414 of the needle cannula 412, while preventing solid particles, such as glass shards, from being drawn into the cap 452 and/or towards the needle cannula 412.

After a sufficient amount of the medical solution is drawn from the ampoule 112 into the syringe barrel 114, the practitioner can remove the needle guard assembly 410 from the ampoule 112 and can move the arms 472, 474 from the extended or initial position to the retracted position. For example, the practitioner can press against the arms 472, 474 causing the arms 472, 474 to move away from the hooks 484 of the retaining ring 482, as shown in FIG. 13B. Once the arms 472, 474 are released from the hooks 484 of the retaining ring 482, the arms 472, 474 can automatically move towards the retention position, as shown in FIG. 12C. In other examples, the arms 472, 474 may not be biased to the retention position. In that case, the practitioner may actively press on the arms 472, 474 causing the arms 472, 474 to move from the extended position to the retracted position. FIG. 12C shows the needle guard assembly 410 with the arms 472, 474 in the retracted position and the sharpened tip at the distal end 418 of the needle cannula 412 exposed and ready for use.

Once the sharpened tip of the needle cannula 412 is exposed and ready for use (as shown in FIG. 12C), the practitioner can insert the sharpened tip of the needle cannula 412 into the skin, muscular tissue, and/or vasculature of the patient and, as previously described, move the stopper 120 through the syringe barrel 114 in the distal direction (shown by arrow D in FIG. 12C) for expelling the medical solution from the syringe barrel 114 to the patient through the lumen 414 of the needle cannula 412. The syringe 110 is shown with the stopper 120 in its distal-most position in FIG. 12D, after all medical solution has been expelled from the syringe barrel 114 and through the lumen 414 of the needle cannula 412.

As shown in FIG. 12E, after delivery of the medical solution to the patient is complete, the practitioner can cause the arms 472, 474 to move the cap 452 back to the initial or extended position in order to provide a shield or barrier enclosing the sharpened tip of the needle cannula 412. For example, the practitioner can press on the arms 472, 474 with sufficient force to overcome any biasing force that maintains the arms 472, 474 in the retracted position. The practitioner continues to press on the arms 472, 474 until the arms 472, 474 return to the extended or initial position, as shown in FIG. 12E. In some examples, one or both of the arms 472, 474 can be received by the hooks 484 of the retention ring 482 for retaining the arms 472, 474 in the extended position. For example, as shown in FIG. 13C, one of the arms 472, 474 is received between two hooks 484 for holding the arms 472, 474 in the extended or initial position and, in particular, for preventing the arms 472, 474 from returning to the retracted position. Once the arms 472, 474 are in the extended position with the cap 452 positioned over the distal end 418 of the needle cannula 412, the needle assembly 410 and/or syringe 110 connected thereto can be discarded or disposed of in a safe and accepted manner.

While examples of the needle guard assembly, syringes, and methods of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A needle guard assembly, comprising:
a needle cannula defining a lumen comprising a proximal end, a distal end comprising a sharpened tip, and a tubular body extending between the proximal end and the distal end;
a needle hub connected to the proximal end of the needle cannula configured to be mounted to a fluid delivery device to provide fluid communication between the fluid delivery device and the lumen of the needle cannula; and
a liquid-permeable cover member extending over at least the distal end of the needle cannula comprising a proximal end engaged to the needle hub and a closed distal end enclosing the distal tip of the needle cannula.

2. The needle guard assembly of claim 1, wherein the liquid-permeable cover member is configured to filter solid particles from a medical solution before the medical solution is drawn into the lumen of the needle cannula.

3. The needle guard assembly of claim 1, wherein the needle cannula comprises an injection needle for subcutaneous, intramuscular, and/or intravenous injections.

4. The needle guard assembly of claim 1, wherein the cover member is substantially rigid defining a cylindrical cavity enclosing the sharpened tip of the needle cannula without contacting the needle cannula.

5. The needle guard assembly of claim 1, wherein the cover member comprises a plurality of perforations for permitting solutions to pass through the cover member and into the lumen of the needle cannula, and
wherein the perforations comprise holes made by laser cutting or heat staking.

6. The needle guard assembly of claim 5, wherein the plurality of perforations comprise a first plurality of holes extending through a sidewall of the cover member and a second plurality of holes extending through the closed distal end of the cover member.

7. The needle guard assembly of claim 1, further comprising an outer shield configured to be positioned over the cover member for protecting the cover member during transportation and before the use,
wherein the outer shield comprises a proximal portion configured to engage a portion of the outer surface of the needle hub for securing the outer shield to the needle hub, and
wherein the portion of the outer surface of the needle hub contacted by the outer shield is proximal to a portion of outer surface of the needle hub contacted by the cover member.

8. The needle guard assembly of claim 1, wherein the cover member is tiered comprising at least a narrow distal tier and a wide proximal tier, and wherein perforations are on the distal tier, with the proximal tier being free from perforations,
the needle guard assembly further comprising a seal positioned in the cover member comprising a central opening sized to receive a sidewall of the needle cannula.

9. The needle guard assembly of claim 8, wherein the seal is seated at an intersection between the proximal tier and the distal tier and, wherein the seal allows for creation of suction in a space defined by the seal and the cover member during aspiration of medical solution into the lumen of the needle cannula.

10. The needle guard assembly of claim 8, further comprising a removable cap positioned over the perforations of the cover member,
wherein the cap comprises a closed distal end sized to receive the closed distal end of the cover member, an outer proximal end, and a sidewall extending between the distal end and the proximal end, and
wherein the sidewall of the cap contacts an outer surface of the cover member forming a friction engagement with the cover member for retaining the cap on the cover member.

11. The needle guard assembly of claim 1, wherein the cover member comprises a cap comprising an open proximal end, a closed distal end, and a cylindrical sidewall extending therebetween, and
a first arm and a second arm mounted between the needle hub and the cap for moving the cap along the needle cannula between an initial position, where the cap covers the distal end of the needle cannula, and a retracted position where the cap is positioned on a sidewall of needle cannula proximal to the distal end of the needle cannula, thereby exposing the sharpened tip of the needle cannula.

12. The needle guard assembly of claim 11, wherein the cap slides along the needle cannula between the initial position and the retracted position.

13. The needle guard assembly of claim 11, wherein the first arm and the second arm each comprise a proximal segment hingedly joined to a distal segment, and wherein the distal segments bend radially inwardly relative to the proximal segments as the first arm and the second arm move from the initial position to the retracted position.

14. The needle guard assembly of claim 13, wherein the first arm and the second arm are biased to the retracted position, and wherein, in the retracted position, the distal segment of the first arm and/or the second arm is substantially perpendicular to a longitudinal axis of the needle guard.

15. The needle guard assembly of claim 13, further comprising a retention ring positioned about the needle hub configured to engage a portion of the first arm or the second arm for retaining the cap over the distal end of the needle cannula, and
wherein the retaining ring comprises hooks configured to engage the first arm and/or the second arm for maintaining the arms in the initial positon.

16. The needle guard assembly of claim 1, further comprising:
a cylindrical seal positioned at the distal end of the cover member, the cylindrical seal comprising a central channel comprising a proximal portion sized to receive the distal end of the needle cannula and a narrowed distal portion extending to an opening in the closed distal end of the cover member; and
a filter positioned between the cylindrical seal and the closed distal end of the cover member, positioned so that medical solution pirated through the cover member passes through the filter and then into the lumen of the needle cannula.

17. The needle guard assembly of claim 1, further comprising an elastomeric ring positioned about an outer surface of the tubular body of the needle cannula,
wherein the cover member comprises at least one slot configured to receive the elastomeric ring for retaining the ring within the cover member as the cover member is removed from the needle cannula, thereby removing the ring from the needle cannula, and
wherein the cover member is configured such that the ring is received within the at least one slot as the cover member is removed from the needle cannula, and the ring slides past the at least one slot without being received in the at least one slot as the needle cannula is inserted into the cover member. 18 A fluid delivery device comprising
a barrel comprising an open proximal end, a closed distal end comprising a distal tip, and a sidewall extending between the proximal end and the distal end;
the needle guard assembly of claim 1, mounted to the distal tip of the barrel such that the lumen of the needle cannula is in fluid communication with an interior of the barrel through the distal tip of the barrel; and
a stopper in the barrel for expelling a medical solution from the barrel through the distal tip and to the lumen of the needle cannula.

19. A method of use for a fluid delivery device comprising the needle guard assembly of claim 1, the method comprising:
inserting the needle guard assembly comprising the cover member and the needle cannula into a medical fluid container;
drawing medical solution in the container through the cover member and into the lumen of the needle cannula;
drawing the medical solution from the lumen of the needle cannula into a reservoir of the fluid delivery device; and
removing the needle guard assembly from the container after a sufficient amount of the medical solution is collected in the reservoir.

20. The method of claim 19, wherein the medical fluid container comprises a glass ampoule containing the medical solution, the method further comprising breaking a narrow neck of the glass ampoule prior to inserting the needle guard assembly into the glass ampoule.
